SEQUENCE LISTING (42 sequences)

SEQ ID NO: 1 - DNA, 4807 bp, Sus scrofa
  misc_feature (426)..(440): n is a, c, g, or t
  misc_feature (2196)..(2297): n is a, c, g, or t
atcataagacttcttggaaaatgaaactgaaaacgggtaagtccctctttctaacctgctcgggcagcgctcgctaagagacgggtgacgggcctggcgggcagggttctgattggcagcctggacgtgggctataaatgcgggcagggagccgagctctcattccaccgccagcaccgctccagtagcgatggctcccctcgtggccttggtcctgctcgggctgctctcactgtctggcctggatgcggtcgcgcgtgagtcctcctcctcccctccctcttccctcttctaccagccctgcctctcccgctcaagccatcgggcactccgggcgctaactctcgggggcactctgggcacgaacgctgggggggtcaggtcccgctcgccaagtttcccttcccggctcggtggacaagcccnnnnnnnnnnnnnnntggagtcggtggggtgggctaggtgtggggaccgggtgcgagtccagcttgtgtgggctcggcggagggaacctctgacacgccggtgcctggtgggaagcggtagactctcggggtgcgggtaagcttcagatcgaccgaggtgggggaggggttctcttccgctgctcggagggacttcaggctccctaggcgcacctggagcagaggacccgctagcgggtcccaaagcgcaggggtggggcgggcgaatgggtggagtcgcccccaagtccaggtctgtcaatcggctgggagcctgagggtcttgagcttcgaccaggcacccgccaggttcagctgcccttctgtactttccccaaatatgtttgccatttaatcgtgaaatgcgtcctcgaggcgaacaagatgtcttatcatcagcacgtcacagatgaggaaagggaggcacgaagagttttgaagtaacttaattacaccggggacaaagggcagacaggggagccagaccggagcatttggcggggttgtgtgccctttggctgtaatccgggcgctctccacatccttctagggtgagattcttgggagcaagctgggcgccatggcccctgttgcctttcttcacagaggacttcctctttggctccttgcctggttctttctgcgatgagctgagtctgctttcagttttgagtaagcgaaggtttatcagtcagaaggtttatcagccgcaaaactccagataggagtaattaacatgctcacccaggctgtctgtcaggacactgtggaactccagtaccttctacatactccgcagtgtatcagaccctcagatttttctcactcacaaaagcctttgtgaagtcgggttttgttttgttttgttttgatcaagagaaaaaggaacttatacgttgttgacaatataaaacattatgtgtgtcatcactgttattgatatttggtagcgactatgtaagtctttcaaacaaacaaacaaacaaacaaactgaggagttcccatggtgaccaggggaaaggaatccgactaggaaccatgaggtggtgggttcaatccctggcctcagtcagtgagttaaggacccgccgttgccgtgagctgtggtgtaggtcgcagatgcagctgcggctgtggtgtaggccagcaactgtagctccagttcgacccccagcctgggaacctccatgtgccacaggtgcagcccccaaaagcaaaaacaaaacaaaacaaaaacaaacaaccccccctcgacatgaaaactatcttaatttgataatttccaaaataatccatgcctggaagatacacaagaatgtttctggaagacatataagaaagtgttaagtgggaatgcctgtggggttgtattttgtgctgtttcctgtgtgtggatttcatttcactgtgggcataagttagttgttcagtttagctatagaaactcagctctcccaaagtcacatagccagcagtggctgaccagattctgctattgctgctttcttggaagagattattgtgatggtcttgcagagaccacaaagagagaactccaggcaggatgaacatgtgttctcatgactttagcatttattatgttctaattactagaagctcaaaagcaaagagatttctgatttatcttcatttttaactatgaaaacatatcagggcttcgtggtatttgcagtctagctcttggtgggactagggaccttttctttgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctgaatgtagctgtatttaaaggtgggttattgacatgtagaagtgtcagtctgtatttttagtagcagtaacacttctctccactttgagagaaacacttagacaactgacagtacttttttcccacatccccatggaggatctagtcatgcatcccatgttggaaaccactgttctacagcattatacaatattgacagcctggcagatgatggcaatcaaccttgacatgctgattaagagtgggaatcccattctcacacacttggccttaatggagagtatgttttctcccatttgctacagtcctcacccttccctgttgttcttacccatcccaactcactcatttaaggtgtttgccagctcctctaggccttttggttctgttcctttgttttctagctcatgaaatcaggtacaaagtcagatttgtcacataggctctggacatagtaaaaaggttgtgttttgtacaaacctactttttttttttttttttttttttgtcttttttgccattttcttgggctgctcccacggcatatggaggttcccaggctaggggtcgaatcggacttgtagccaccggcctatgccagagccacagcaacgcagggtccgagccgtgtctgcaacctacaccacagctcacagcaatgccggatccttaacccactgagcaaaagccagggatcgaacccgcaacctcatggttcctagtcggattcattaaccactgcgccacaacgggaactccaagtacaaacctactttgagttgactctctcaggaacctgagcaaggaggtagtaataacaacacctgccacttacagaacaattgctgtgtgccaggcagtgtactaggcacataccgagaggtctgttggatgtggatctaggcaagaggcaaaaatgaaaggagtaaacataaggaagaaaactgacggggctgaaaacagggcacaataatgtgagttttttgatgtggctactctggaaggttgtgcagtgagaaatgaagcatgagaagtaggtgggaccaaatcatgcagacgctggagtgagcacgaattcagtgtctgctgaggagccatgagaagctcttgggagcctcacagaaaggtccttaacgatttggcacaatctccactgacagaaaaagatgttactagaaatctgctagaaaaaaagcctaaaaggcctgtgtaggggaactgtgtgggcaagtcactacgtcagggtttatccctcaaaacggaggctgtggagttcctgctgtggtgcaacggaaacgaatccgactggtaaccatgaggttgggagttcaatccctggccaagctcagtgggttaaggatctggtgttaccatgagctgtggtgtaggccagcagctacaccacagaccgggctccgatcctgcattgctgtggctgtggtgtaagccagcagctacagctcccaattcaacccctagcctggcaacctccatatgctgcaatgtgtaaaatggaagctcatttggcctgaagggaaatgggctgggagaaatggatggccaagtttaaacacttggtgcctgacctggctttagacacaggtagcccccggtgaaatcctctggcgtttcatctgtctttccttgatgttcctcaggtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggatcagtatagctgccgcgtgactctcgataagcccaagatagttaagtggggtgagttttcaagttctttccttcactgctgccaaccgaatctgagcatagccacagcagaacgctgctctgacctaaaagcatctgcatcctgggattatcagggactttattaaagctccgtctagtagcaccttctgagagacctctgcttggcacagtgcctgagacagtggtctccctgcagtgagagcaaggagcagcagcagcagcagcagcagcacttgcacaagtgcacatgcacttccaacagttcttccctagtggcttctttggctttcctgacagcctcggggagagtcaggaccaaggagagtctcagggaagcacaggccttggtctgtaaggccctgctctcctggtgccctccaatcctggacccctccagttgtctggctggggtgggatctaggccaccaggcaaggttgggtcctaacaaggcgctcatggtcagggacggggggctctcctgccagttgtattataacagtttcagacacttgttagtacacagtatttcaaaagtaaactttaactgttttcctttctccattttcttttgcatagatcgagaccactaa

SEQ ID NO: 2 - DNA, 4823 bp, Sus scrofa
  misc_feature (15)..(105): n is a, c, g, or t
  misc_feature (373)..(436): n is a, c, g, or t
  misc_feature (2193)..(2294): n is a, c, g, or t
atcataagacttagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgattggcagcctggacgtgggctataaatgcgggcagggagccgagctctcattccaccgccagcaccgctccagtagcgatggctcccctcgtggccttggtcctgctcgggctgctctcactgtctggcctggatgcggtcgcgcgtgagtcctcctcctcccctccctcttccctcttctaccagccctgcctctcccgctcaagccatcgggcactccgggcgctaactctgggggcactctgggcacgaacgctggggggnnnnnnnnnnnnnnnnnnnnnnnnnnnnnnnnnnnnnnnnnnnnnnnnnnnnnnnnnnnnnnnntggagtcggtggggtgggctaggtgtggggaccgggtgcgagtccagcttgtgtgggctcggcggaggggaacctctgacacgccggtgcctggtgggaagcggtagactctcggggtgcgggtaagcttcagatcgaccgaggtgggggaggggttctcttccgctgctcggagggacttcaggctccctaggcgcacctggagcagaggacccgctagcgggtcccaaagcgcaggggtggggcgggcgaatgggtggagtcgcccccaagtccaggtctgtcaatcggctgggagcctgagggtcttgagcttcgaccaggcacccgccaggttcagctgcccttctgtactttccccaaatatgtttgccatttaatcgtgaaatgcgtcctcgaggcgaacaagatgtcttatcatcagcacgtcacagatgaggaaagggaggcacgaagagttttgaagtaacttaattacaccggggacaaagggcagacaggggagccagaccggagcatttggcggggttgtgtgccctttggctgtaatccgggcgctctccacatccttctagggtgagattcttgggagcaagctgggcgccatggcccctgttgcctttcttcacagaggacttcctctttggctccttgcctggttctttctgcgatgagctgagtctgctttcagttttgagtaagcgaaggtttatcagtcagaaggtttatcagccgcaaaactccagataggagtaattaacatgctcacccaggctgtctgtcaggacactgtggaactccagtaccttctacatactccgcagtgtatcagaccctcagatttttctcactcacaaaagcctttgtgaagtcgggttttgttttgttttgttttgatcaagagaaaaaggaacttatacgttgttgacaatataaaacattatgtgtgtcatcactgttattgatatttggtagtgactatgtaagcctttcaaacaaacaaacaaacaaacaaactgaggagttcccatggtgaccaggggaaaggaatccgactaggaaccatgaggtggtgggttcaatccctggcctcagtcagtgagttaaggacccgccgttgccgtgagctgtggtgtaggtcgcagatgcagctgcggctgtggtgtaggccagcaactgtagctccagttcgacccccagcctgggaacctccatgtgccacaggtgcagcccccaaaagcaaaaacaaaacaaaacaaaaacaaacaaccccccctcgacatgaaaactatcttaatttgataatttccaaaataatccatgcctggaagatacacaagaatgtttctggaagacatataagaaagtgttaagtgggaatgcctgtggggttgtattttgtgctgtttcctgtgtgtggatttcatttcactgtgggcataagttagttgttcagtttagctatagaaactcagctctcccaaagtcacatagccagcagtggctgaccagattctgctattgctgctttcttggaagagattattgtgatggtcttgcagagaccacaaagagagaactccaggcaggatgaacatgtgttctcatgactttagcatttattatgttctaattactagaagctcaaaagcaaagagatttctgatttatcttcatttttaactatgaaaacatatcagggcttcgtggtatttgcagtctagctcttggtgggactagggaccttttctttgatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctgaatgtagctgtatttaaaggtgggttattgacatgtagaagtgtcagtctgtatttttagtagcagtaacacttctctccactttgagagaaacacttagacaactgacagtacttttttcccacatccccatggaggatctagtcatgcatcccatgttggaaaccactgttctacagcattatacaatattgacagcctggcagatgatggcaatcaaccttgacatgctgattaagagtgggaatcccattctcacacacttggccttaatggagagtatgttttctcccatttgctacagtcctcacccttccctgttgttcttacccatcccaactcactcatttaaggtgtttgccagctcctctaggccttttggttctgttcctttgttttctagctcatgaaaattcaaggtacaaagtcagatttgtcaacataggcttctggacataagtaaaaaggttgtcgttttgcatacaaaccttactttttttttttttttttttttttgtcttttttgccattttcttgggctgctcccacggcatatggaggttcccaggctaggggtcgaatcggacttgtagccaccggcctatgccagagccacagcaacgcagggtccgagccgtgtctgcaacctacaccacagctcacagcaatgccggatccttaacccactgagcaaaagccagggatcgaacccgcaacctcatggttcctagtcggattcattaaccactgcgccacaacgggaactccaagtacaaacctactttgagttgactctctcaggaacctgagcaaggaggtagtaataacaacacctgccacttacagaacaattgctgtgtgccaggcagtgtactaggcacataccgagaggtctgttggatgtggatctaggcaagaggcaaaaatgaaaggagtaaacataaggaagaaaactgacggggctgaaacagggcacaataatgtgagttttttgatgtggctactctggaaggttgtgcagtgagaaatgaagcatgagaagtaggtgggaccaaatcatgcagacgctggagtgagcacgaattcagtgtctgctgaggagccatgagaagctcttgggagcctcacagaaaggtccttaacgatttggcacaatctccactgacagaaaaagatgttactagaaatctgctagaaaaaaagcctaaaagggcctgtgtaggggaactgtgtgggcaagtcactacgtcagggtttatccctcaaaacggaggctgtggagttcctgctgtggtgcaacggaaacgaatccgactggtaaccatgaggttgggagttcaatccctggccaagctcagtgggttaaggatctggtgttaccatgagctgtggtgtaggccagcagctacaccacagaccgggctccgatcctgcattgctgtggctgtggtgtaagccagcagctacagctcccaattcaacccctagcctggcaacctccatatgctgcaatgtgtaaaaatggaagctcatttggcctgaagggaaatgggctgggagaaatggatggccaagtttaaacacttggtgcctgacctggctttagacacaggtagcccccggtgaaatcctctggcgtttcatctgtctttccttgatgttcctcaggtcccccgaaggttcaggtttactcacgccacccagcggaaaacggaaagccaaattacctgaactgctatgtatctgggttccatccgccccagattgaaattgatttgctgaaaacggggagaagatgaacgcggagcagtcagacctgtctttcagcaaggactggtctttctaccttctggtccacactgagttcactcctaacgctgtggatcagtatagctgccgcgtggaagcacgtgactctcgataagcccaagatagttaagtggggtgagttttcaagttctttccttcactgctgccaaccgaatctgagcatagccacagcagaacgctgctctgacctaaaagcatctgcatcctgggattatcagggactttattaaagctccgtctagtagcaccttctgagagacctctgcttggcacagtgcctgagacagtggtctccctgcagtgagagcaaggagcagcagcagcagcagcagcagcacttgcacaagtgcacatgcacttccaacagttcttccctagtggcttctttggctttcctgacagcctcggggagagtcaggaccaaggagagtctcagggaagcacaggccttggtctgtaagccctgctctcctggtgccctccaatcctggacccctccagttgtctggctggggtgggatctaggccaccaggcaaggttgggtcctaacaaggcgctcatggtcagggacggggggctctcctgccagttgtattataacagtttcagacacttgttagtacacagtatttcaaaagtaaactttaactgttttcctttctccattttcttttgcatagatcgagaccactaa

SEQ ID NO: 3 - DNA, 4814 bp, Sus scrofa
  misc_feature (644)..(651): n is a, c, g, or t
  misc_feature (1401)..(1413): n is a, c, g, or t
  misc_feature (4174)..(4316): n is a, c, g, or t
atcataagacttcttggaaaatgaaactgaaaacgggtaagtccctctttctaacctgctcgggcagcgctcgctaagagacgggtgacgggcctggcgggcagggttctgattggcagcctggacgtgggctataaatgcgggcagggagccgagctctcattccaccgccagcaccgctccagtagcgatggctcccctcgtggccttggtcctgctcgggctgctctcactgtctggcctggatgcggtcgcgcgtgagtcctcctcctcccctccctcttccctcttctaccagccctgcctctcccgctcaagccatcgggcactccgggcgctaactctgggggcactctgggcacgaacgctgggggggtcaggtcccgctcgccaagtttcccttcccggctcggtggacaagccccgggcttcctcgctgtggagtcggtggggtgggctaggtgtggggaccgggtgcgagtccagcttgtgtgggctcggcggagggaacctctgacacgccggtgcctggtgggaagcggtagactctcggggtgcgggtaagcttcagatcgaccgaggtgggggaggggttctcttccgctgctcggagggacttcaggctccctaggcgcacctggagnnnnnnnnccgctagcgggtcccaaagcgcaggggtggggcgggcgaatgggtggagtcgcccccaagtccaggtctgtcaatcggctgggagcctgagggtcttgagcttcgaccaggcacccgccaggttcagctgcccttctgtactttccccaaatatgtttgccatttaatcgtgaaatgcgtcctcgaggcgaacaagatgtcttatcatcagcacgtcacagatgaggaaagggaggcacgaagagttttgaagtaacttaattacaccggggacaaagggcagacaggggagccagaccggagcatttggcggggttgtgtgccctttggctgtaatccgggcgctctccacatccttctagggtgagattcttgggagcaagctgggcgccatggcccctgttgcctttcttcacagaggacttcctctttggctccttgcctggttctttctgcgatgagctgagtctgctttcagttttgagtaagcgaaggtttatcagtcagaaggtttatcagccgcaaaactccagataggagtaattaacatgctcacccaggctgtctgtcaggacactgtggaactccagtaccttctacatactccgcagtgtatcagaccctcagatttttctcactcacaaaagcctttgtgaagtcgggttttgttttgttttgttttgatcaagagaaaaaggaacttatacgttgttgacaatataaaacattatgtgtgtcatcactgttatnnnnnnnnnnnnncgactatgtaagtctttcaaacaaacaaacaaacaaacaaactgaggagttcccatggtgaccaggggaaaggaatccgactaggaaccatgaggtggtgggttcaatccctggcctcagtcagtgagttaaggacccgccgttgccgtgagctgtggtgtaggtcgcagatgcagctgcggctgtggtgtaggccagcaactgtagctccagttcgacccccagcctgggaacctccatgtgccacaggtgcagcccccaaaagcaaaaacaaaacaaaacaaaaacaaacaaccccccctcgacatgaaaactatcttaatttgataatttccaaaataatccatgcctggaagatacacaagaatgtttctggaagacatataagaaagtgttaagtgggaatgcctgtggggttgtattttgtgctgtttcctgtgtgtggatttcatttcactgtgggcataagttagttgttcagtttagctatagaaactcagctctcccaaagtcacatagccagcagtggctgaccagattctgctattgctgctttcttggaagagattattgtgatggtcttgcagagaccacaaagagagaactccaggcaggatgaacatgtgttctcatgactttagcatttattatgttctaattactagaagctcaaaagcaaagagatttctgatttatcttcatttttaactatgaaaacatatcagggcttcgtggtatttgcagtctagctcttggtgggactagggaccttttctttgatgatggggtgtctctaggcaggtacatctggtcattgaccaggtactacccacctccccaccttctgaacatggaggaagagatgacctttataaccatgtaccctgaatgtagctgtatttaaaggtgggttattgacatgtagaagtgtcagtctgtatttttagtagcagtaacacttctctccactttgagagaaacacttagacaactgacagtacttttttcccacatccccatggaggatctagtcatgcatcccatgttggaaaccactgttctacagcattatacaatattgacagcctggcagatgatggcaatcaaccttgacatgctgattaagagtgggaatcccattctcacacacttggccttaatggagagtatgttttctcccatttgctacagtcctcacccttccctgttgttcttacccatcccaactcactcatttaaggtgtttgccagctcctctaggccttttggttctgttcctttgttttctagctcatgaaatcaggtacaaagtcagatttgtcacataggctctggacatagtaaaaaggttgtgttttgtacaaacctactttttttttttttttttttttttgtcttttttgccattttcttgggctgctcccacggcatatggaggttcccaggctaggggtcgaatcggacttgtagccaccggcctatgccagagccacagcaacgcagggtccgagccgtgtctgcaacctacaccacagctcacagcaatgccggatccttaacccactgagcaaaagccagggatcgaacccgcaacctcatggttcctagtcggattcattaaccactgcgccacaacgggaactccaagtacaaacctactttgagttgactctctcaggaacctgagcaaggaggtagtaataacaacacctgccacttacagaacaattgctgtgtgccaggcagtgtactaggcacataccgagaggtctgttggatgtggatctaggcaagaggcaaaaatgaaaggagtaaacataaggaagaaaactgacggggctgaaacagggcacaataatgtgagttttttgatgtggctactctggaaggttgtgcagtgagaaatgaagcatgagaagtaggtgggaccaaatcatgcagacgctggagtgagcacgaattcagtgtctgctgaggagccatgagaagctcttgggagcctcacagaaaggtccttaacgatttggcacaatctccactgacagaaaaagatgttactagaaatctgctagaaaaaaagcctaaaaggcctgtgtaggggaactgtgtgggcaagtcactacgtcagggtttatccctcaaaacggaggctgtggagttcctgctgtggtgcaacggaaacgaatccgactggtaaccatgaggttgggagttcaatccctggccaagctcagtgggttaaggatctggtgttaccatgagctgtggtgtaggccagcagctacaccacagaccgggctccgatcctgcattgctgtggctgtggtgtaagccagcagctacagctcccaattcaacccctagcctggcaacctccatatgctgcaatgtgtaaaaatggaagctcatttggcctgaagggaaatgggctgggagaaatggatggccaagtttaaacacttggtgcctgacctggctttagacacaggtagcccccggtgaaatcctctggcgtttcatctgtctttccttgatgttcctcaggtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctgcatcctgggattatcagggactttattaaagctccgtctagtagcaccttctgagagacctctgcttggcacagtgcctgagacagtggtctccctgcagtgagagcaaggagcagcagcagcagcagcagcagcacttgcacaagtgcacatgcacttccaacagttcttccctagtggcttctttggctttcctgacagcctcggggagagtcaggaccaaggagagtctcagggaagcacaggccttggtctgtaagccctgctctcctggtgccctccaatcctggacccctccagttgtctggctggggtgggatctaggccaccaggcaaggttgggtcctaacaaggcgctcatggtcagggacggggggctctcctgccagttgtattataacagtttcagacacttgttagtacacagtatttcaaaagtaaactttaactgttttcctttctccattttcttttgcatagatcgagaccactaa

SEQ ID NO: 4 - DNA, 4817 bp, Sus scrofa
  misc_feature (641)..(648): n is a, c, g, or t
  misc_feature (4177)..(4319): n is a, c, g, or t
atcataagacttagaaaatgaaactgaaaacgggttggtccctcattctaacctgctcgggcagcgctcgctaagagacgggtgacgggcctggcgggcagggttctgattggcagcctggacgtgggctataaatgcgggcagggagccgagctctcattccaccgccagcaccgctccagtagcgatggctcccctcgtggccttggtcctgctcgggctgctctcactgtctggcctggatgcggtcgcgcgtgagtcctcctcctcccctccctcttccctcttctaccagccctgcctctcccgctcaagccatcgggcactccgggcgctaactctgggggcactctgggcacgaacgctgggggggtcaggtcccgctcgccaagtttcccttcccggctcggtggacaagccccgggcttcctcgctgtggagtcggtggggtgggctaggtgtggggaccgggtgcgagtccagcttgtgtgggctcggcggagggaacctctgacacgccggtgcctggtgggaagcggtagactctcggggtgcgggtaagcttcagatcgaccgaggtgggggaggggttctcttccgctgctcggagggacttcaggctccctaggcgcacctggagnnnnnnnnccgctagcgggtcccaaagcgcaggggtggggcgggcgaatgggtggagtcgcccccaagtccaggtctgtcaatcggctgggagcctgagggtcttgagcttcgaccaggcacccgccaggttcagctgcccttctgtactttccccaaatatgtttgccatttaatcgtgaaatgcgtcctcgaggcgaacaagatgtcttatcatcagcacgtcacagatgaggaaagggaggcacgaagagttttgaagtaacttaattacaccggggacaaagggcagacaggggagccagaccggagcatttggcggggttgtgtgccctttggctgtaatccgggcgctctccacatccttctagggtgagattcttgggagcaagctgggcgccatggcccctgttgcctttcttcacagaggacttcctctttggctccttgcctggttctttctgcgatgagctgagtctgctttcagttttgagtaagcgaaggtttatcagtcagaaggtttatcagccgcaaaactccagataggagtaattaacatgctcacccaggctgtctgtcaggacactgtggaactccagtaccttctacatactccgcagtgtatcagaccctcagatttttctcactcacaaaagcctttgtgaagtcgggttttgttttgttttgttttgatcaagagaaaaaggaacttatacgttgttgacaatataaaacattatgtgtgtcatcactgttattgatatttggtagtgactatgtaagcctttcaaacaaacaaacaaacaaacaaactgaggagttcccatggtgaccaggggaaaggaatccgactaggaaccatgaggtggtgggttcaatccctggcctcagtcagtgagttaaggacccgccgttgccgtgagctgtggtgtaggtcgcagatggcagctgcggctgtggtgtaggccagcaactgtagctccagttcgacccccagcctgggaacctccatgtgccacaggtgcagccccccaaaaagcaaaaaacaaaaacaaaaacaaaaacaaacaaccccccctcgacatgaaaactatcttaatttgataatttccaaaataatccatgcctggaagatacacaagaatgtttctggaagacatataagaaagtgttaagtgggaatgcctgtggggttgtattttgtgctgtttcctgtgtgtggatttcatttcactgtgggcataagttagttgttcagtttagctatagaaactcagctctcccaaagtcacatagccagcagtggctgaccagattctgctattgctgctttcttggaagagattattgtgatggtcttgcagagaccacaaagagagaactccaggcaggatgaacatgtgttctcatgactttagcatttattatgttctaattactagaagctcaaaagcaaagagatttctgatttatcttcatttttaactatgaaaacatatcagggcttcgtggtatttgcagtctagctcttggtgggactagggaccttttctttgatgatggggtgtctctaggcaggtacatctggtcattgaccaggtactacccacctccccaccttctgaacatggaggaagagatgacctttataaccatgtaccctgaatgtagctgtatttaaaggtgggttattgacatgtagaagtgtcagtctgtatttttagtagcagtaacacttctctccactttgagagaaacacttagacaactgacagtacttttttcccacatccccatggaggatctagtcatgcatcccatgttggaaaccactgttctacagcattatacaatattgacagcctggcagatgatggcaatcaaccttgacatgctgattaagagtgggaatcccattctcacacacttggccttaatggagagtatgttttctcccatttgctacagtcctcacccttccctgttgttcttacccatcccaactcactcatttaaggtgtttgccagctcctctaggccttttggttctgttcctttgttttctagctcatgaaatcaggtacaaagtcagatttgtcacataggctctggacatagtaaaaaggttgtgttttgtacaaacctactttttttttttttttttttttttgtcttttttgccattttcttgggctgctcccacggcatatggaggttcccaggctaggggtcgaatcggacttgtagccaccggcctatgccagagccacagcaacgcagggtccgagccgtgtctgcaacctacaccacagctcacagcaatgccggatccttaacccactgagcaaaagccagggatcgaacccgcaacctcatggttcctagtcggattcattaaccactgcgccacaacgggaactccaagtacaaacctactttgagttgactctctcaggaacctgagcaaggaggtagtaataacaacacctgccacttacagaacaattgctgtgtgccaggcagtgtactaggcacataccgagaggtctgttggatgtggatctaggcaagaggcaaaaatgaaaggagtaaacataaggaagaaaactgacggggctgaaacagggcacaataatgtgagttttttgatgtggctactctggaaggttgtgcagtgagaaatgaagcatgagaagtaggtgggaccaaatcatgcagacgctggagtgagcacgaattcagtgtctgctgaggagccatgagaagctcttgggagcctcacagaaaggtccttaacgatttggcacaatctccactgacagaaaaagatgttactagaaatctgctagaaaaaaagcctaaaaggcctgtgtaggggaactgtgtgggcaagtcactacgtcagggtttatccctcaaaacggaggctgtggagttcctgctgtggtgcaacggaaacgaatccgactggtaaccatgaggttgggagttcaatccctggccaagctcagtgggttaaggatctggtgttaccatgagctgtggtgtaggccagcagctacaccacagaccgggctccgatcctgcattgctgtggctgtggtgtaagccagcagctacagctcccaattcaacccctagcctggcaacctccatatgctgcaatgtgtaaaaatggaagctcatttggcctgaagggaaatgggctgggagaaatggatggccaagtttaaacacttggtgcctgacctggctttagacacaggtagcccccggtgaaatcctctggcgtttcatctgtctttccttgatgttcctcaggtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctgcatcctgggattatcagggactttattaaagctccgtctagtagcaccttctgagagacctctgcttggcacagtgcctgagacagtggtctccctgcagtgagagcaaggagcagcagcagcagcagcagcagcacttgcacaagtgcacatgcacttccaacagttcttccctagtggcttctttggctttcctgacagcctcggggagagtcaggaccaaggagagtctcagggaagcacaggccttggtctgtaagccctgctctcctggtgccctccaatcctggacccctccagttgtctggctggggtgggatctaggccaccaggcaaggttgggtcctaacaaggcgctcatggtcagggacggggggctctcctgccagttgtattataacagtttcagacacttgttagtacacagtatttcaaaagtaaactttaactgttttcctttctccattttcttttgcatagatcgagaccactaa

SEQ ID NO: 5 - protein, 119 aa, Artificial Sequence
  HLA-B2M Donor 11, 19, 29, 50, and 57
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 6 - protein, 118 aa, Artificial Sequence
  HLA-B2M XT-PAM Cell
MAPLVALVLLGLLSLSGLDAVARPPKVQVYSRHPAENGKPNYLNCYVSGFHPPQIEIDLLKNGEKMNAEQSDLSFSKDWSFYLLVHTEFTPNAVDQYSCRVKHVTLDKPKIVKWDRDH

SEQ ID NO: 7 - RNA, 20 nt, Artificial Sequence
  pB2m KO Guide RNA Sequence
cgagagucacgugcuucacg

SEQ ID NO: 8 - DNA, 18 bp, Artificial Sequence
  Porcine B2M Control Sequence Copy 1
tgaagcacgtgactctcg

SEQ ID NO: 9 - DNA, 10 bp, Artificial Sequence
  Human B2M Knock-Out Sequence Copy 2
tgactctcga

SEQ ID NO: 10 - DNA, 20 bp, Artificial Sequence
  Porcine B2M Control Sequence Copy 2
gccgcgtgaagcacgtgact

SEQ ID NO: 11 - DNA, 21 bp, Artificial Sequence
  Human B2M Knock-Out Sequence Copy 2
gccgcgtggaagcacgtgact

SEQ ID NO: 12 - protein, 12 aa, Artificial Sequence
  Human B2m Peptides (17-28)
GLEAIQRTPKIQ

SEQ ID NO: 13 - protein, 12 aa, Artificial Sequence
  Porcine B2M Peptides (17-28)
GLDAVARPPKVQ

SEQ ID NO: 14 - protein, 10 aa, Artificial Sequence
  Human B2M Peptides (64-73)
ERIEKVEHSD

SEQ ID NO: 15 - protein, 10 aa, Artificial Sequence
  Porcine B2M Peptides (64-73)
EKMNAEQSDL

SEQ ID NO: 16 - protein, 10 aa, Artificial Sequence
  Human B2M Peptides (91-100)
TPTEKDEYAC

SEQ ID NO: 17 - protein, 10 aa, Artificial Sequence
  Porcine B2M Peptides (90-99)
TPNAVDQYSC

SEQ ID NO: 18 - protein, 118 aa, Artificial Sequence
  Human B2M Cross-reactive Epitope groups
SRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 19 - protein, 117 aa, Artificial Sequence
  Porcine B2M Cross-reactive Epitope Groups
APLVALVLLGLLSLSGLDAVARPPKVQVYSRHPAENGKPNYLNCYVSGFHPPQIEIDLLKNGEKMNAEQSDLSFSKDWSFYLLVHTEFTPNAVDQYSCRVKHVTLDKPKIVKWDRDH

SEQ ID NO: 20 - DNA, 199 bp, Artificial Sequence
  Porcine Promoter Region 5′-3′
catcataagacttcttggaaaatgaaactgaaaacgggtaagtccctctttctaacctgctcgggcagcgctcgctaagagacgggtgacgggcctggcgggcagggttctgattggcagcctggacgtgggctataaatgcgggcagggagccgagctctcattccaccgccagcaccgctccagtagcgatggctcc

SEQ ID NO: 21 - DNA, 199 bp, Artificial Sequence
  Porcine Promoter Region 3′-5′
gtagtattctgaagaaccttttactttgacttttgcccattcagggagaaagattggacgagcccgtcgcgagcgattctctgcccactgcccggaccgcccgtcccaagactaaccgtcggacctgcacccgatatttacgcccgtccctcggctcgagagtaaggtggcggtcgtggcgaggtcatcgctaccgagg

SEQ ID NO: 22 - DNA, 606 bp, Artificial Sequence
  Rosa26 Region of PAM cell line (promoter)
  misc_feature (409)..(450): n is a, c, g, or t
gggcagcatcctcctgctgagagccgggggagggccaggcccacgtcccgagagcaagcgcgaggagacggaggaggtgacccttccctcccccggggcccggtggtgaggggaggtctctcttttctgtcgcacccttaccttgtcccaggcctgggcccgggctgcggcgcacggcactcccggtaggcagcaggactcgagttaggcccagcgcggcgccacggcgtttcctggccgggaatggcccgtgcccgtgaggtgggggtggggggcaaaaaggcggagcgagccaaaggcggtgagggggagggccagggaaggaggggggggccggcactactgtgttggcggactggcgggactggggctgcgtgagtctctgagcgcaggcgggcggcggcgcccnnnnnnnnnnnnnnnnnnnnnnnnnnnnnnnnnnnnnnnnnnctcactcagcccgctgcccgagcggaaacgccactgaccgcacggggattcccagcgccggcgccaggggcacccgggacacgccccctcccgccgcgccattggcccctccgcccaccgtctcgcacccattggccagctccccgccaatcagcg

SEQ ID NO: 23 - DNA, 710 bp, Artificial Sequence
  Rosa26 Region of PAM cell line (Exon 1)
gaagccgccggggccgcctagagaagaggctgtgctctggggctccggctcctcagagagcctcggctaggtaggggagcgggactctggtttgggggagggccggcggtttggcgggggatgggtgcttgaggtggtctgaccggtagcgggggtcgccttccctagcgggaagtcgggagcatatcgtttgttacgctggaaggggaagaggtggtgagaggcaggcgggagtgcggcccgccctgcggcaaccggagggggagggagaagggagcggaaaagcctggaatacggacggagccattgctcccgcagagggaggagcgcttcctgctcttctcttgtcactgattggccgcttctcctcccgccgtgtgtgaaaacacaaatggcgtgttttggttggagtaaagctcctgtcagttacagcctcgggagtgcgcagcctcccaggaactctcgcattgccccctgggtgggtaggtaggtggggtggagagagctgcacaggcgggcgctgtcggcctcctgcggggggaggggagggtcagtgaaagtggctcccgcgcgggcgtcctgccaccctcccctccgggggagtcggtttacccgccgcctgctcggctttggtatctgattggctgctgaagtcctgggaacggccccttgttattggcttgggtcccaaatgagcgaaaccactacgc

SEQ ID NO: 24 - DNA, 576 bp, Artificial Sequence
  Rosa26 Region of PAM cell line (Exon 2)
ttttaatttctagtatggtaaaatactggtaaacaaagcatttgggaccctcagcttttaataatgtgaagatatcctgagaccaagaagttggaggaagctgctaagcatagtgagtatacatggtaaacactcaaatgcctgttgaatagctggatgtagtgaaaaatgactccaggtattaagcctggattactgggagggtgggcatgccaggacagggagcagattttaatggagaaagataggttcaagttgagttttaagtaggacaggcaggggtaatcacaggaggcagctggaaattcagaatttgggcttgagaaaactaaaataccatgttttcctttccagtgaaaattgctttcatcatagactatatgattgaatagctacaatcccacagctgattagcatacatgtttgccttggggcagaagtatgagggtgacaaagcaggcttctcataagcaatggtaaattttttcacattgtgcacattaaaaaatgcagtataggagagagctgtctatatggtgttcctcttcaacttaaaaataaataaataaccaaggt

SEQ ID NO: 25 - DNA, 109 bp, Artificial Sequence
  Donor B2M KO at Rosa26
atgtcttggaaaaaggctttgcggatccccggaggccttcgggcagcaactgttaccttgatgctggcgatgctgagcaccccagtggctgagggcagagactctcccg

SEQ ID NO: 26 - DNA, 84 bp, Artificial Sequence
  Human B2m KI at Rosa26
aggctttgcggatccccggaggccttcgggcagcaactgttaccttgatgctggcgatgctgagcaccccagtggctgagggca

SEQ ID NO: 27 - DNA, 550 bp, Artificial Sequence
  Complete B2M (swine promoter and human exons 1, 2, and 3)
atcataagacttcttggaaaatgaaactgaaaacgggtaagtccctctttctaacctgctcgggcagcgctcgctaagagacgggtgacgggcctggcgggcagggttctgattggcagcctggacgtgggctataaatgcgggcagggagccgagctctcattccaccgccagcaccgctccagtagcgatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 28 - DNA, 353 bp, Artificial Sequence
  pB2M Rosa26
cgatggctcccctcgtggccttggtcctgctcgggctgctctcactgtctggcctggatgcggtcgcgcgtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggatcagtatagctgccgcgtgaagcacgtgactctcgataagcccaagatagttaagtgggatcgagac

SEQ ID NO: 29 - DNA, 356 bp, Artificial Sequence
  Humanized pB2M 5′-3′
cgatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagac

SEQ ID NO: 30 - DNA, 356 bp, Artificial Sequence
  Humanized pB2M 3′-5′
gctacagagcgaggcaccggaatcgacacgagcgcgatgagagagaaagaccggacctccgataggtcgcatgaggtttctaagtccaaatgagtgcagtaggtcgtctcttacctttcagtttaaaggacttaacgatacacagacccaaagtaggtaggctgtaacttcaactgaatgacttcttacctctctcttaactttttcacctcgtaagtctgaacagaaagtcgttcctgaccagaaagatagagaacatgatgtgacttaagtgggggtgactttttctactcatacggacggcacacttggtacactgaaacagtgtcgggttctatcaattcaccctagctctg

SEQ ID NO: 31 - DNA, 15 bp, Artificial Sequence
  Porcine B2M Control Sequence Copy 1
gaagcacgtgactct

SEQ ID NO: 32 - DNA, 15 bp, Artificial Sequence
  Porcine B2M Control Sequence Copy 2
acggggagaagatga

SEQ ID NO: 33 - DNA, 14 bp, Artificial Sequence
  Porcine B2M Knock-Out Sequence Copy 2 (5′-3′)
cggggagaagatga

SEQ ID NO: 34 - DNA, 14 bp, Artificial Sequence
  Porcine B2M Knock-Out Sequence Copy 2 (3′-5′)
gcccctcttctact

SEQ ID NO: 35 - DNA, 357 bp, Artificial Sequence
  Porcine B2M Coding Region 5′-3′
atggctcccctcgtggccttggtcctgctcgggctgctctcactgtctggcctggatgcggtcgcgcgtcccccgaaggttcaggtttactcacgccacccagcggaaaacggaaagccaaattacctgaactgctatgtatctgggttccatccgccccagattgaaattgatttgctgaaaaacggggagaagatgaacgcggagcagtcagacctgtctttcagcaaggactggtctttctaccttctggtccacactgagttcactcctaacgctgtggatcagtatagctgccgcgtgaagcacgtgactctcgataagcccaagatagttaagtgggatcgagaccactaa

SEQ ID NO: 36 - DNA, 357 bp, Artificial Sequence
  Porcine B2M Coding Region 3′-5′
taccgaggggagcaccggaaccaggacgagcccgacgagagtgacagaccggacctacgccagcgcgcagggggcttccaagtccaaatgagtgcggtgggtcgccttttgcctttcggtttaatggacttgacgatacatagacccaaggtaggcggggtctaactttaactaaacgactttttgcccctcttctacttgcgcctcgtcagtctggacagaaagtcgttcctgaccagaaagatggaagaccaggtgtgactcaagtgaggattgcgacacctagtcatatcgacggcgcacttcgtgcactgagagctattcgggttctatcaattcaccctagctctggtgatt

SEQ ID NO: 37 - protein, 118 aa, Artificial Sequence; identical to SEQ ID NO: 6
  Porcine B2M Coding Region Amino Acids

SEQ ID NO: 38 - DNA, 360 bp, Artificial Sequence
  Human B2M Coding Region 5′-3′
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 39 - DNA, 360 bp, Artificial Sequence
  Human B2M Coding Region 3′-5′
tacagagcgaggcaccggaatcgacacgagcgcgatgagagagaaagaccggacctccgataggtcgcatgaggtttctaagtccaaatgagtgcagtaggtcgtctcttacctttcagtttaaaggacttaacgatacacagacccaaagtaggtaggctgtaacttcaactgaatgacttcttacctctctcttaactttttcacctcgtaagtctgaacagaaagtcgttcctgaccagaaagatagagaacatgatgtgacttaagtgggggtgactttttctactcatacggacggcacacttggtacactgaaacagtgtcgggttctatcaattcaccctagctctgtacatt

SEQ ID NO: 40 - protein, 119 aa, Artificial Sequence; identical to SEQ ID NO: 5
  Human B2M Coding Region Amino Acids

SEQ ID NO: 41 - protein, 89 aa, Artificial Sequence
  Porcine Edited copy 1 (A2 line)
RPPKVQVYSRHPAENGKPNYLNCYVSGFHPPQIEIDLLKNGEKMNAEQSDLSFSKDWSFYLLVHTEFTPNAVDQYSCRVTLDKPKIVKW

SEQ ID NO: 42 - protein, 91 aa, Artificial Sequence
  Porcine edited copy 2 (A2 line)
RPPKVQVYSRHPAENGKPNYLNCYVSGFHPPQIEIDLLKTGRRTRSSQTCLSARTGLSTFWSTLSSLLTLWISIAAAWKHVTLDKPKIVKW

(19) United States
(12) Patent Application Publication
(10) Pub. No.: US 2026/0114433 A1
(43) Pub. Date: Apr. 30, 2026

(54) HUMANIZATION OF BETA2-MICROGLOBULIN IN PORCINE GENOME RESULTING IN FUNCTIONAL EXPRESSION OF HUMAN BETA2-MICROGLOBULIN WITHIN DONOR CELLS, TISSUES, OR ORGANS

(71) Applicants: Xenotherapeutics, Inc., Grantham, NH (US); Alexis Bio, Inc., Grantham, NH (US)

(72) Inventors: Paul W. HOLZER, Grantham, NH (US); Rodney L. MONROY, North Fort Myers, FL (US); Elizabeth J. CHANG, Pittsford, NY (US); Kaitlyn ROGERS, Madisonville, LA (US)

(73) Assignees: Xenotherapeutics, Inc., Grantham, NH (US); Alexis Bio, Inc., Grantham, NH (US)

(21) Appl. No.: 18/257,671

(22) PCT Filed: Dec. 16, 2021
(86) PCT No.: PCT/US2021/063931

§ 371 Date: Jun. 15, 2023

Related U.S. Application Data
(60) Provisional application No. 63/126,254, filed on Dec. 16, 2020.

(51) Int. Cl. A01K 67/0275; C07K 14/74

(52) U.S. Cl. CPC A01K 67/0275; C07K 14/70539; A01K 2207/15; A01K 2217/072; A01K 2217/075; A01K 2217/15; A01K 2227/108; A01K 2267/025; C07K 2319/21

(57) ABSTRACT

A method of modifying copy (1) and copy (2) of the β2-microglobulin (β2M) gene of a porcine donor animal, genetically modified porcine donor animal wherein copy (1) and copy (2) of the β2M gene of the porcine donor animal genome are modified, and a method of producing a donor porcine donor animal tissue or organ for xenotransplantation, wherein copy (1) and copy (2) of the β2M gene of the cells of said donor porcine donor animal are modified. A method of humanizing a genetically engineered donor animal by modification of the donor animal's expressed native β2-microglobulin protein through genetic alterations of the endogenous β2M genes, and as a result, the newly expressed β2M protein of the donor animal would contain some or all of the amino acids of the orthologous human β2-microglobulin (hβ2M) protein, in identity and orientation. This results in immune-compatible xenotransplants between donor animals and human recipients.

### CROSS REFERENCE TO RELATED APPLICATION

The present application claims priority benefit of U.S. 63/126,254 filed on Dec. 16, 2020.

### TECHNICAL FIELD

The present disclosure relates to humanizing a genetically engineered donor animal by modification of the donor animal's expressed native β2M protein through genetic alterations of the endogenous β2M genes.

### BACKGROUND

Xenotransplantation from donor animals could help realize the therapeutic potential of interchangeable, functionally-equivalent, immune-compatible, cross-species cells, tissues, and organs.

However, wild-type donor animals require genetic modification(s) to prevent or delay immunological rejection mechanisms by the recipient's immune response. These can be categorized as hyperacute, acute, and chronic rejection.

Hyperacute rejection is a result of the innate immune system's response via pre-formed antibodies against endothelial α1,3-galactosyl-galactose (αGal) epitopes, resulting in complement activation and rapid graft destruction via ischemia. All animals, with the exception of Old-World primates, present α1,3-galactosyl-galactose (αGal) epitopes on the surface of all nucleated cells.

Inactivation of the GGTA1 (alpha-galactosyltransferase 1) gene results in the reduction of this innate immune response, prolonging survival of the xenotransplant. However, additional genetic modification(s) are required to further reduce the residual innate immune response mechanisms, as well as cell-mediated immunological rejection processes, resulting in immune-compatible xenotransplants between donor animals and human recipients.

The major histocompatibility complex (MHC) Class I proteins are highly polymorphic extracellular molecules which play a major role in the immune system of all vertebrates, and are a chief target of the rejection response after organ transplantation.

MHC-I molecules heterotrimeric complex consisting of four distinct extracellular alpha domains, two of which have thousands of allelic variants and one is highly conserved intra-species, and the remaining domain is comprised of a light β-chain, the β2M protein.

Functional expression of MHC-I molecules requires this component. As a result, disruption of the β2M protein provides an effective method to eliminate MHC-1 expression on the cell surface.

The complete hβ2M gene is approximately 8 kilobases (kb) in size, the clinical relevance of this 30-kDa protein is well characterized. Elevated serum β2M concentrations have been observed in many pathological conditions, including renal disease, immunodeficiency, and autoimmune diseases. Further, β2M concentrations have been reported as elevated at the time of diagnosis in many solid and hematological cancers. β2M deficient mice are known to be more susceptible to intracellular pathogens than wild-type.

β2M is believed to arise in a basal jawed vertebrate (gnathostome). β2M protein sequences are highly conserved within species, and overall structure is highly similar as many structural amino acid residues and orientations are conserved. This includes two characteristically spaced cysteine residues which form the disulfide bridge. The secondary structure of β2M consists of seven β-strands which are organized into two β-sheets linked by a single disulfide bridge, presenting a classical β-sandwich typical of the immunoglobulin (Ig) domain.

However, numerous non-conserved amino acid substitutions exist inter-species. The hβ2M shows 70% amino acid sequence similarity to the murine version and the responsible genes are located on syntenic chromosomes. hβ2M also has a 73% amino acid sequence similarity to the porcine β2M (pβ2M).

No genetic variant of hβ2M is known, an evolutionary rare event suggesting that changes in the β2M protein sequence could be undesirable to the host. T-cell recognition of extracellular foreign protein sequences of non-hβ2M create antigenic regions that are recognizable to antibodies, B- and T-cells. This results in cell mediated, direct and indirect rejection mechanisms. Further, since β2M does not have a transmembrane domain, if segments of β2M that are bonded with the three alpha domains of MHC-I are disrupted, secretion of free-β2M protein into circulation results in indiscriminate systemic adverse immune cascades.

In contrast to humans, the porcine-β2M protein (pβ2M)gene is approximately 45.5 kb, due to an identical duplication of the β2M gene (copy 1 and copy 2) on the chromosome 1, separated by a unique intronic region. This causes surface expression of the pβ2M variant 12 times greater than the surface expression of the hβ2M protein variant (hβ2M) in humans, although the respective contributions from the individual copies are believed to be unlikely in equal proportions. This segmental duplication of pβ2M gene might occur through the participation of repeat elements, especially long interspersed nuclear elements (LINE) which are located on the edges of duplicated gene blocks at a rate of 2 to 3 times higher than the average. Moreover, it has been reported that evolutionary breakpoint regions (EBRs) are enriched for the LINE elements.

To the inventors' knowledge, this phenomenon occurs only in cetartiodactyl and Sus scrofa species. Researchers posit the presence of the duplicate gene copies is likely to enhance the capacity of the animals' immune systems.

The inventors' experience and analysis of this genetic region includes sequencing of the identical copy 1 and copy 2 of the pβ2M from unpublished experimental cell lines. The inventors have demonstrated that the identical sequence and participation of repeat elements in the pβ2M segmental duplication causes design of guide RNAs (gRNAs) necessary to target genetic modifications to select only one copy of pβ2M gene (i.e. only copy 1 or only copy-2) to be functionally impossible with conventional technology.

Thus, any genetic alteration to this region is necessarily of a higher complexity than those that would be otherwise equivalent. Thus, there remains a need to develop methods to address the inter-species disparity in the β2M protein.

There is a need for cross-species cell interchangeable methods for humanizing the β2M protein of a donor animal via genetic alteration of the donor animal β2M gene(s).

### SUMMARY OF THE INVENTION

The present disclosure describes multiplexed genomic editing of the non-human donor animal, e.g., porcine, genome to insert an immune-compatible β2M gene.

The present disclosure includes the humanization of the pβ2M protein via a site-directed mutagenic substitution, and in some aspects, the scarless exchange, of regions of pβ2M gene to the genome of the porcine donor animal, resulting in a functional, extracellularly expressed β2M protein that confers enhanced immune-compatibility between cells, tissues, organs derived from porcine donor animals and human recipients.

In the present disclosure, the disclosures of US2020/0283737A1 are incorporated herein by reference as if entirely recited herein for all purposes.

In one aspect, the present disclosure includes a method including some or all of the following steps:
1. Obtaining a multipotent or pluripotent cell, from a wild-type non-human donor animal reared in a Designated Pathogen Free (DPF) environment. Such cells may include, for example, fibroblasts, Mesenchymal Stem Cells (pMSCs), bone marrow cells, zygotes, induced pluripotent stem cells (IPSCs), or germ-line cells. Cell populations selected may operate under AAALAC, OLAW, and/or USDA oversight, with procedures performed in compliance with FDA Current Good Manufacturing Practice (cGMP) regulations;
2. Before, during, or after other genetic alterations, copy 1 and copy 2 of the pβ2M gene are inactivated from the porcine genome;
3. Before, during, or after other genetic alterations, a genomic sequence construct is created that includes forming a genomic sequence construct that comprises a) a nucleic acid sequence encoding a 5′ UTR upstream region of a hβ2M gene or a pβ2M gene including its promoter sequence and b) a nucleic acid sequence encoding a hβ2M protein;
4. The hβ2M gene construct described above is inserted into the safe harbor locus ROSA26 or any other safe harbor locus of the porcine genome.

In one aspect, the present disclosure includes a method of generating a genetically reprogrammed non-human donor animal comprising:
a) obtaining a multipotent or pluripotent cell from a wild-type non-human donor animal, wherein said multipotent or pluripotent cell comprise a porcine genome comprising copy 1 and copy 2 of a porcine β2-microglobulin (pβ2M) gene;
b) inactivating copy 1 and/or copy 2 of the pβ2M gene in the porcine genome;
c) forming a genomic sequence construct that comprises a) a nucleic acid sequence encoding a 5′ UTR upstream region of a hβ2M gene or a pβ2M gene including its promoter sequence and b) a nucleic acid sequence encoding a hβ2M protein;
d) performing scarless exchange of a nucleotide acid sequence encoding copy 1 and/or copy 2 of the pβ2M gene in the porcine genome with the genomic sequence construct of step c) to form a genetically reprogrammed cell;
e) generating an embryo from the genetically reprogrammed cell;
f) transferring the embryo into a surrogate animal; and
g) growing the transferred embryo to produce the genetically reprogrammed non-human donor animal as offspring of said surrogate animal.

A 5′ UTR is a region at the 5′ end of a mature transcript (preceding the initiation codon) that is not translated into a protein. In some aspects, the 5′ UTR of the pβ2M gene (copy 1) is 190 base pairs. In some aspects, the 5′ UTR including the species-specific promoter sequence may have a sequence of SEQ ID NO: 20.

In some aspects, the hβ2M gene is 360 base pairs.

The present disclosure includes a genomic sequence construct that comprises a) a nucleic acid sequence encoding a 5′ UTR upstream region of a hs2M gene or a pβ2M gene including its promoter sequence and b) a nucleic acid sequence encoding a hβ2M protein.

In one aspect, humanization of pβ2M is performed following additional genetic alterations to the wild-type porcine genome which reduce the natural immunologic response to xenotransplants between donor animals and human recipients.

In one aspect, the present disclosure includes human and porcine B2M Cross-reactive Epitope Groups (CREGs) Predictive Analysis to assess whether a B2m mismatch within a CREG group may result in a better outcome than a mismatch outside CREG groups. The process includes analyzing validated outcomes data against known amino acid characteristics.

In one aspect, the present disclosure includes a donor porcine donor animal tissue or organ for xenotransplantation obtained from a genetically modified porcine donor animal, wherein the cells from the genetically modified porcine donor animal when co-cultured with human peripheral blood mononuclear cells (PBMCs) induce a lower CD8+ T cell immune response as compared to cells from said non-genetically modified counterpart pig, as measured by an in vitro mixed lymphocyte reaction assay.

In one aspect, the insertion of a hβ2M gene construct in safe harbor locus ROSA26 of the porcine genome allows for gene expression without interrupting the function of essential endogenous genes.

In one aspect, the present disclosure includes a method of modifying copy 1 and copy 2 of the pβ2M gene.

In one aspect, the present disclosure includes a genetically modified porcine donor animal wherein copy 1 and copy 2 of the pβ2M gene of the porcine donor animal genome are modified.

In one aspect, the present disclosure includes a method of producing a donor porcine donor animal tissue or organ for xenotransplantation, wherein copy 1 and copy 2 of the β2M gene of the cells of said donor porcine donor animal are modified.

In one aspect, the present disclosure includes a method of generating a genetically modified porcine donor wherein copy 1 and copy 2 of the pβ2M gene are modified.

### BRIEF DESCRIPTION OF THE DRAWINGS

The accompanying drawings, which are incorporated herein and form part of the disclosure, help illustrate various aspects of the present invention and, together with the description, further serve to describe the invention to enable a person skilled in the pertinent art to make and use the aspects disclosed herein. In the drawings, like reference numbers indicate identical or functionally similar elements.

FIGS. 1A-1B illustrate characterization of PAM cells wherein the porcine pulmonary alveolar cell line (clone 34D/21) was used.

FIG. 2 shows phenotyping analysis of PAM cells. The PAM cells were cultured in medium alone (control) or were activated for 72 hours with 100 ng/mL IFN-γ or loaded 30 μg/mL KLH for 24 hours. The cells were stained for SLA-DQ, and marker is detected using anti mouse APC-conjugated polyclonal IgG secondary antibody. Data is presented as histograms of count (y axis) versus fluorescence intensity in log scale (x axis). Percentage of positive and negative cells for SLA-DQ for activated cells are shown on histograms.

FIG. 3A shows 2-dimensional and 3-dimensional protein structures of the MHC-antigen peptide complex for MHC Class I. FIG. 3B shows MHC Class I molecules wherein B2M is present on all six MHC Class I Isotypes in both the human and porcine proteins.

FIG. 4 shows a comparison of amino acid sequences of Beta-2-Microglobulin among porcein PAM cells and humans.

FIG. 5A shows the location of Human B2m on Chromosome 15. FIG. 5B shows the location of Porcine B2m on Chromosome 1.

FIG. 6 shows schematic depiction of an immune-compatible porcine cell according to the present disclosure.

FIG. 7 illustrates key areas of antigenicity in Human and Porcine B2m proteins

FIG. 8 illustrates human and porcine B2M cross-reactive epitope groups (CREGs) predictive analysis.

FIG. 9 illustrates human and porcine B2M cross-reactive epitope groups (CREGs) predictive analysis to assess whether a B2m mismatch within a CREG group may result in a better outcome than a mismatch outside CREG groups using validated outcomes data against known amino acid characteristics.

FIG. 10 shows a production process for recombinant monoclonal antibody production.

FIG. 11 shows gel electrophoresis of porcine B2M recombinant protein.

FIG. 12A shows 24-well plate template for antigen loaded donor cell generation.

FIG. 12B shows 96-well V-bottom plate template for antigen loaded DC generation of KLH. FIG. 12C shows 96-well V-bottom plate template for antigen loaded DC generation of hB2M. FIG. 12D shows 96-well V-bottom plate template for antigen loaded DC generation of pB2M.

FIG. 13A shows immunogenicity response of autologous human B2M loaded dendritic cells. FIG. 13B shows immunogenicity response of autologous porcine B2M loaded dendritic cells.

FIG. 14 shows Naïve T-cells enrichment of modified PAM cells with porcine B2M Copy 1 & 2 Knock-out.

FIG. 15 shows genomic mapping of porcine B2M with region designation key.

FIG. 16 shows schematic of porcine promoter region.

FIG. 17A shows a schematic of a porcine B2M coding region. FIG. 17B shows a schematic of a human B2M coding region.

FIG. 18A demonstrates the steps for gene-editing: (1) A knockout of the porcine B2M Copy 1 and Copy 2 using a large fragment deletion at exon 2; (2) Insertion of B2M from a designated human donor was placed into the Rosa26 safe harbor site. Light grey is used to indicate a knockout or fragdel has occurred, blue is representative of a human donor knock-in of the gene.

FIG. 18B the steps for gene-editing: (1) A knockout of the porcine B2M Copy 1 and Copy 2 using a large fragment deletion at exon 2; (2) Insertion of B2M from a designated human donor was placed into either Copy 1 or Copy 2. Light grey is used to indicate a knockout or fragdel has occurred, blue is representative of a human donor knock-in of the gene.

FIG. 19 shows pB2M KO Guide RNA Sequence and Cut Locations.

FIG. 20A shows the map of Porcine B2M Copy 1, including exon 2, where a 9 base pair (bp) edit was knocked out of Porcine B2M Copy 1. FIG. 20B shows the map of Porcine B2M Copy 2, including exon 2, where a base pair (bp) edit was knocked out of Porcine B2M Copy 2.

FIG. 21 shows viability of the PAM clone cells determined using trypan blue dye exclusion method.

FIG. 22 shows β2M protein expression in the genetically modified PAM cells.

FIG. 23A shows Flow Histograms for cell surface B2M and SLA Class I molecules of WT-Clone A8. FIG. 23B shows Flow Histograms for cell surface B2M and SLA Class I molecules of Clone A9-Clone B5. FIG. 23C shows Flow Histograms for cell surface B2M and SLA Class I molecules of B6-Clone C4. FIG. 23D shows Flow Histograms for cell surface B2M and SLA Class I molecules of C5-Clone C12.

FIG. 24 shows identification of two ideal B2M KO clones.

FIG. 25A shows comprehensive A2 B2M copy 1 sequence. FIG. 25B shows the comprehensive A2 clone B2M copy 2 sequence. FIG. 25C shows the comprehensive A5 clone B2M copy 1 sequence. FIG. 25D shows the comprehensive A5 clone B2M copy 2 sequence.

FIG. 26 shows Simulating Agarose Gel Electrophoresis in SnapGene comparing Sanger sequencing from the WT PAM cell to the porcine b2m fragdel edits on PAM cell A2. Within the perspective gel model, lane 1 is porcine B2m sequence, lane 2 is A2 copy 1 exons only, lane 3 is A2 copy 2 exons only.

FIG. 27 shows human B2M Knock In at the Rosa26 safe harbor site.

FIG. 28 shows complete B2m sequences comprising porcine promoter and human exons 1, 2, 3 upon Human B2M KI at Rosa26 safe harbor site

FIG. 29 shows possible permutations of hB2M KI construct for insertion into Rosa26 site.

FIG. 30 shows hB2M Knock In Construct for insertion into the ROSA26 site.

FIG. 31A shows junction PCR of Human B2M KI at Porcine Rosa26 safe harbor site wherein the clones show modest B2M expression. FIG. 31B shows junction PCR attempt on clones that are puromycin resistant. Clones show modest B2M expression, with Clones 10, 13, 14, and 15 showing best expression

FIG. 32 shows flow cytometry results demonstrating B2M expression wherein Clones 10, 13, 14, and 15 exhibit the strongest expression of hB2M on the cell surface.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. Other features and advantages of the invention will be apparent from the following detailed description and figures, and from the claims.

The singular forms “a,” “an,” and “the” include plural referents unless the context clearly dictates otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

“Designated pathogen free,” and its grammatical equivalents as used herein include reference to animals, animal herds, animal products derived therefrom, and/or animal facilities that are free of one or more specified pathogens. Preferably, such “designated pathogen free” animals, animal herds, animal products derived therefrom, and/or animal facilities are maintained using well-defined routines of testing for such designated pathogens, utilizing proper standard operating procedures (SOPs) and practices of herd husbandry and veterinary care to assure the absence and/or destruction of such designated pathogens, including routines, testing, procedures, husbandry, and veterinary care disclosed and described herein. It will be further understood that as used herein the term “free,” and like terms when used in connection with “pathogen free” are meant to indicate that the subject pathogens are not present, not alive, not active, or otherwise not detectable by standard or other testing methods for the subject pathogens. Pathogens can also include, but not be limited to, emerging infectious diseases that have newly appeared in a population or have existed but are rapidly increasing in incidence or geographic range, or that are caused by one of the United States National Institute of Allergy and Infectious Diseases (NIAID) Category A, B, or C priority pathogens.

“Alter,” “altering,” “altered” and grammatical equivalents as used herein include any and/or all modifications to a gene including, but not limited to, deleting, inserting, silencing, modifying, reprogramming, disrupting, mutating, rearranging, increasing expression, knocking-in, knocking out, and/or any or all other such modifications or any combination thereof.

“Endogenous loci” and its grammatical equivalents as used herein include the natural genetic loci found in the animal to be transformed into the donor animal.

“Functional,” e.g., in reference to a functional polypeptide, and its grammatical equivalents as used herein include a polypeptide that retains at least one biological activity normally associated with the native protein. For example, in some embodiments, a replacement at an endogenous locus (e.g., replacement at an endogenous non-human MHC I, MHC II, and/or Beta-2-Microglobulin (B2M) locus) results in a locus that fails to express a functional endogenous polypeptide. Likewise, the term “functional” as used herein in reference to the functional extracellular domain of a protein, can refer to an extracellular domain that retains its functionality, e.g., in the case of MHC I, ability to bind an antigen, ability to bind a T-cell co-receptor, etc. In some embodiments, a replacement at the endogenous MHC locus results in a locus that fails to express an extracellular domain (e.g., a functional extracellular domain) of an endogenous MHC while expressing an extracellular domain (e.g., a functional extracellular domain) of a human MHC.

“Genetic or molecular marker,” and their grammatical equivalents as used herein include polymorphic locus, i.e., a polymorphic nucleotide (a so-called single nucleotide polymorphism or SNP) or a polymorphic DNA sequence at a specific locus. A marker refers to a measurable, genetic characteristic with a fixed position in the genome, which is normally inherited in a Mendelian fashion, and which can be used for mapping of a trait of interest. Thus, a genetic marker may be a short DNA sequence, such as a sequence surrounding a single base-pair change, i.e., a single nucleotide polymorphism or SNP, or a long DNA sequence, such as microsatellites or Simple Sequence Repeats (SSRs). The nature of the marker is dependent on the molecular analysis used and can be detected at the DNA, RNA, or protein level. Genetic mapping can be performed using molecular markers such as, but not limited to, RFLP (restriction fragment length polymorphisms; Botstein et al. (1980), Am J Hum Genet. 32:314-331; Tanksley et al. (1989), Bio/Technology 7:257-263), RAPD [random amplified polymorphic DNA; Williams et al. (1990), NAR 18:6531-6535], AFLP [Amplified Fragment Length Polymorphism; Vos et al. (1995) NAR 23:4407-4414], SSRs or microsatellites [Tautz et al. (1989), NAR 17:6463-6471]. Appropriate primers or probes are dictated by the mapping method used.

“Improving” and its grammatical equivalents as used herein include any improvement recognized by one of skill in the art. For example, improving transplantation can mean lessening hyperacute rejection, which can encompass a decrease, lessening, or diminishing of an undesirable effect or symptom. In some aspects, a clinically relevant improvement is achieved.

“Locus” (loci plural) or “site” and their grammatical equivalents as used herein include a specific place or places on a chromosome where, for example, a gene, a genetic marker, or a QTL is found.

“Capture sequence” or “reference sequence” and their grammatical equivalents as used herein include a nucleic acid or amino acid sequence that has been obtained, sequenced, or otherwise become known from a sample, animal (including humans), or population. For example, a capture sequence from a human patient is a “human patient capture sequence.” A capture sequence from a particular human population is a “human population-specific human capture sequence.” And a capture sequence from a human allele group is an “allele-group-specific human capture sequence.”

“Humanized” and its grammatical equivalents as used herein include embodiments wherein all or a portion of an endogenous non-human gene or allele is replaced by a corresponding portion of an orthologous human gene or allele. For example, in some embodiments, the term “humanized” refers to the complete replacement of the coding region (e.g., the exons) of the endogenous non-human MHC gene or allele or fragment thereof with the corresponding capture sequence of the human MHC gene or allele or fragment thereof, while the endogenous non-coding region(s)(such as, but not limited to, the promoter, the 5′ and/or 3′ untranslated region(s), enhancer elements, etc.) of the non-human animal donor is not replaced.

“Reprogram,” “reprogrammed,” including in reference to “immunogenomic reprogramming,” and their grammatical equivalents as used herein, refer to the replacement or substitution of endogenous nucleotides in the donor animal with orthologous nucleotides based on a separate reference sequence, wherein frameshift mutations are not introduced by such reprogramming. In addition, reprogramming results in no net loss or net gain in the total number of nucleotides in the donor animal genome, or results in a net loss or net gain in the total number of nucleotides in the donor animal genome that is equal to no more than 1%, no more than 2%, no more than 3%, no more than 4%, no more than 5%, no more than 6%, no more than 7%, no more than 8%, no more than 9%, no more than 10%, no more than 12%, no more than 15%, or no more than 20% of the number of nucleotides in the separate reference sequence. In one example of “reprogramming,” an endogenous non-human nucleotide, codon, gene, or fragment thereof is replaced with a corresponding synthetic nucleotide, codon, gene, or fragment thereof based on a human capture sequence, through which the total number of base pairs in the donor animal sequence is equal to the total number of base pairs of the human capture sequence.

“Tolerogenic” and its grammatical equivalents as used herein include characteristics of an organ, cell, tissue, or other biological product that are tolerated by the reduced response by the recipient's immune system upon transplantation.

“Transgenic” and its grammatical equivalents as used herein, include donor animal genomes that have been modified to introduce non-native genes from a different species into the donor animal's genome at a non-orthologous, non-endogenous location such that the homologous, endogenous version of the gene (if any) is retained in whole or in part. “Transgene,” “transgenic,” and grammatical equivalents as used herein do not include reprogrammed genomes, knock in/knockouts, site-directed mutagenic substitutions or series thereof, or other modifications as described and claimed herein. By way of example, “transgenic” porcine donor include those having or expressing hCD46 (“human membrane cofactor protein,” or “MCP”), hCD55 (“human decay-accelerating factor,” “DAF”), human Beta-2-Microglobulin (B2M), and/or other human genes, achieved by insertion of human gene sequences at a non-orthologous, non-endogenous location in the porcine donor genome without the replacement of the endogenous versions of those genes.

In organ, tissue, and stem cell transplantation, one challenge in successful transplantation is to find a host and a donor with tissue types as similar as possible. Accordingly, in organ, tissue, and stem cell transplantation, the key to success is finding a host and a donor with tissue types as similar as possible. Histocompatibility, or tissue compatibility, is the property of having the same or sufficiently similar alleles of the MHC such that the recipient's MHC does not trigger the immune system to reject the donor's tissue.

In transplantation, MHC molecules act themselves as antigens, provoking an immune response from a recipient, leading to transplant rejection. Accordingly, eliminating the expression of specific MHC molecules from the donor will serve to reduce immunological rejection of transplanted porcine cells, tissues, and/or organs, into a human recipient. However, complete elimination of MHC molecules may also result in rejection due to innate immune response. Human MHC Class I and II are also called human leukocyte antigen (HLA). For the donor animals to survive and thrive, it is necessary to retain certain MHC molecules (e.g., SLAs) that provide the donor animals with a minimally competent immune system. Prior art strategies that rely on the deletion of the MHC gene pose significant risks to the donor animals, e.g., severe combined immune deficiency (SCID). Prior art strategies that do not reprogram the porcine genome pose significant risks of rejection to the human recipient or require significant and endless use of immunosuppressants.

Because MHC variation in the human population is very high, it has been difficult or impossible to obtain cells, tissue, or organs for xenotransplantation that express MHC molecules sufficiently identical to the recipient for safe and effective transplantation of organs and tissues. Further, diversity and amino acid variations in non-MHC molecules between human and porcine are a cause of immunological rejection of wild-type porcine cells. The immunoreactivity of xenograft may vary with natural variations of MHC in the donor population. On the other hand, natural variation in human MHC also modulates the intensity of immune response.

β2M is found on the surface of most nucleated cells as part of a heterodimer molecule with MHC class I expression that attaches to a cell surface. The presence of β2M plays a key role in the immunological rejection process of xenotransplants between donor animals and human recipients. β2M is a beta light chain of human leukocyte antigen class I molecule (HLA-I). Its main function is to participate in the recognition of lymphocytes and target cell surface antigens, so β2M is closely related to histocompatibility. Deletion of β2M leads to an abnormal polymerization of HLA-I molecules, which cannot form intact functional molecules. The ROSA26 gene locus is one region of the genome that provides a “safe harbor” for transgene expression without interrupting the function of essential endogenous genes. The sequence of porcine ROSA26 (pROSA26) locus has been completely characterized, and the pRosa26 promoter has been identified. Therefore, porcine ROSA26 could be targeted efficiently and support abundant ubiquitous transgene expression.

The promoter of the pβ2M gene is suitable for driving exogenous gene expression in a high and stable manner by avoiding DNA methylation. Thus, the use of the upstream regions of the pβ2M gene including the species-specific promoter sequence to drive expression of an immune-compatible, e.g., humanized, or a porcine-human hybrid, β2M protein has been successfully employed by the inventors in the present disclosure.

The present disclosure relates to humanizing a genetically engineered donor animal by modification of the donor animal's expressed native β2M protein through genetic alterations of the endogenous β2M genes, and as a result, the newly expressed β2M protein of the donor animal would contain some or all of the amino acids of the orthologous hβ2M protein, in identity and orientation.

The present disclosure also relates to the transposition of genetic sequences responsible for the expression of the β2M protein from their native, wild-type chromosomal location to other locations in the genome, including safe harbor loci, such as ROSA26.

According to the present disclosure, the genetic modification can be made utilizing known genome editing techniques, such as zinc-finger nucleases (ZFNs), transcription activator-like effector nucleases (TALENs), adeno-associated virus (AAV)-mediated gene editing, and clustered regular interspaced palindromic repeat Cas9 (CRISPR-Cas9).

These programmable nucleases enable the targeted generation of DNA double-stranded breaks (DSB), which promote the upregulation of cellular repair mechanisms, resulting in either the error-prone process of non-homologous end joining (NHEJ) or homology-directed repair (HDR), the latter of which is used to integrate exogenous donor DNA templates. CRISPR-Cas9 may also be used to perform precise modifications of genetic material.

For example, the genetic modification via CRISPR-Cas9 can be performed in a manner described in Kelton, W. et. al., “Reprogramming MHC specificity by CRISPR-Cas9-assisted cassette exchange,” Nature, Scientific Reports, 7:45775 (2017) (“Kelton”), where the entire disclosure of which is incorporated herein by reference. Accordingly, the present disclosure includes reprogramming using CRISPR-Cas9 for site-directed mutagenic substitutions to mediate rapid and scarless exchange of pβ2M gene to hβ2M gene.

Alternatively or in conjunction to duplication, the hβ2M gene may be attached to a porcine donor animal promoter sequence providing higher affinity to porcine transcription factors and forming a genomic sequence construct that comprises a) a nucleic acid sequence encoding a 5′ UTR upstream region of a hβ2M gene or a pβ2M gene including its promoter sequence and b) a nucleic acid sequence encoding a hβ2M protein. The sequence may optionally include a spacer. In some aspects, a Kozak consensus sequence is placed at the 5′, between 5′UTR and hB2M as the spacer. The 5′ UTR may be a human 5′ UTR sequence or a donor animal 5′ UTR sequence.

According to the present disclosure, the other genetic alteration may include any or all modifications aimed to reduce the recipients' natural immunologic response(s), including but not limited to, modification to PERV, MHC Class I, MHC Class II, alpha-1,3 galactosyltransferase, cytidine monophosphate-N-acetylneuraminic acid hydroxylase (CMAH), β1,4-N-acetylgalactosaminyltransferase, PD-L1, CTLA-4, Endothelial protein C receptor (EPCR), Thrombomodulin (TBM), and Tissue Factor Pathway Inhibitor (TFPI).

It will be understood that any number of non-human donor animals could be utilized in accordance with the present invention, including, but not limited to, pigs, non-human primates, monkeys, sheep, goats, mice, cattle, deer, horses, dogs, cats, rats, mules, and any other mammals. Source animals could also include any other animals including, but not limited to, birds, fish, reptiles, and amphibians.

It will be further understood that any animal serving as a source animal hereunder, may be configured, engineered, or otherwise altered and/or maintained, may be created, bred, propagated and/or maintained in accordance with the present disclosure to create and maintain animals and resulting biological products to be used in or in preparation or pursuit of clinical xenotransplantation.

For example, the present disclosure includes non-human animals, e.g., porcine, having certain combinations of specific genetic characteristics, breeding characteristics and the specifically recited pathogen-free profile. Such animals may include, as described above and herein, immunogenomic reprogrammed porcine having a biologically reprogrammed genome such that it does not express one or more extracellular surface glycan epitopes, e.g., genes encoding alpha-1,3 galactosyltransferase, cytidine monophosphate-N-acetylneuraminic acid hydroxylase (CMAH), and β1,4-N-acetylgalactosaminyltransferase are disrupted such that surface glycan epitopes encoded by said genes (alpha-1,3-galactose, Neu5Gc, and Sia-alpha2,3-[GalNAc-beta1,4]Gal-beta1,4-GlcNAc (SD^{a}), respectively) are not expressed, as well as other modifications to the porcine's SLA to express MHC-I or MHC-II, and regulation of PD-1 and CTLA4, as described above and herein. Resulting from the process described herein, the porcine is free of at least the following zoonotic pathogens:
(i) Ascaris species, cryptosporidium species, Echinococcus, Strongyloids sterocolis, and Toxoplasma gondii in fecal matter;
(ii) Leptospira species, Mycoplasma hyopneumoniae, porcine reproductive and respiratory syndrome virus (PRRSV), pseudorabies, transmissible gastroenteritis virus (TGE)/a coronavirus, Toxoplasma gondii in antibody titers;
(iii) Porcine Influenza;
(iv) the following bacterial pathogens as determined by bacterial culture: Bordetella bronchisceptica, Coagulase-positive staphylococci, Coagulase-negative staphylococci, Livestock-associated methicillin resistant Staphylococcus aureus (LA MRSA), Microphyton and Trichophyton spp.;
(v) Porcine cytomegalovirus; and
(vi) Brucella suis;
   is raised and maintained according to a bioburden-reducing procedure, the procedure comprising maintaining the porcine in an isolated closed herd, wherein all other animals in the isolated closed herd are confirmed to be free of said zoonotic pathogens; wherein the porcine is isolated from contact with any non-human animals and animal housing facilities outside of the isolated closed herd.

In another aspect it will be understood that there includes an adventitious agent control strategy developed based on the source animal, including the species, strain, geographic origin, type of tissue, and proposed indication. Analytical Tests are conducted for adventitious agents, to include bacteria, fungi, mycoplasma, and viral microorganisms, including as follows:
a. Bacteriological Free Status—The bacteriological screen is conducted to confirm the drug product is free of potential biological agents of concern Humans. Both Aerobic and Anaerobic screens are conducted to ensure sterility. Samples are thawed as described herein and transferred to Tryptic Soy Broth (TSB) or Fluid Thioglycollate Medium (FTM) as appropriate. Vessels will be incubated to allow for potential growth. If no evidence of microbial growth is found, the product will be judged to comply with the test for sterility.
b. Mycological (Fungal) Free Status—The mycological screen is conducted to confirm the Drug Product is free of potential fungal agents of concern. Samples are thawed as described herein. After thawing, samples are transferred to a soybean-casein digest agar. Vessels will be incubated to allow for potential growth. If no evidence of fungal growth is found, the product will be judged to comply with the test for sterility per USP<71>.
c. Mycoplasma Free Status—The mycoplasma screen is conducted to confirm the drug product is free of mycoplasma. Samples are thawed as described herein and added to 100 mL of Mycoplasma broth and incubated at 37° C. for up to 21 days. The sample is sub-cultured after 2-4 days, 7-10 days, 14 days, and 21 days. The plates are then incubated at 37° C. for up to 14 days and checked for the presence of Mycoplasma colonies. If none are detected, the product is found to be in compliance with USP<63> and is mycoplasma free.
d. Endotoxin Free Status—The endotoxin free status is conducted to confirm the drug product is free of endotoxins and related agents of concern. Three samples from the same lot will be tested for the Inhibition/Enhancement of the Limulus amoebocyte lysate (LAL) test. Samples will be thawed as described herein and extracted with 40 mL of WFI per sample at 37° C. for 1 hour. Samples will then be tested in the LAL Kinetic Chromogenic Test with a standard curve ranging from 5-50EU/mL at a validated dilution. Assays will be performed in compliance with USP<85>.
e. Viral Assays Conducted—The viral assays are conducted to confirm the source animal is free of potential viral agents of concern, confirmation of endogenous viruses (see below). This includes co-culturing and RT-PCR testing for specific latent endogenous viruses including PERV. In vivo assays are also conducted on the animal source to monitor animal health and freedom from viral infection as key aspects of the lot release criteria. Due to the endemic nature of PERV in porcine tissue, this qualifies as a positive result that does not preclude the use of such tissue. However, the virus is identified and characterized in lot release to provide information for monitoring the recipient of the xenotransplantation product.
f. Cell Viability Assay—The MTT assay is conducted to confirm the biologically active status of cells in the xenotransplantation product. Evidence of viability is provided through surrogate markers of mitochondrial activity as compared to positive (fresh, not cryopreserved) and negative (heat-denatured) controls. The activity of the cells is required for the xenotransplantation product to afford the intended clinical function. This is required as a lot release criteria, and is currently established that tissue viability should not be less than 50% of the metabolic activity demonstrated by the fresh tissue control comparator.
g. Histology and Morphology—Verification under microscope via visible examination of Hematoxylin and Eosin (H&E) section staining of the epidermal and dermal layers, to ensure correct cell morphology and organization of the xenotransplantation product tissues and cell infiltrate populations. This is conducted to confirm the appropriate physiologic appearance and identity of cells present in the xenotransplantation product. The xenotransplantation product is composed of porcine dermal and epidermal tissue layers. This is required as a lot release criteria. Evidence of the following cell layers (from most superficial to deepest), in the epidermal layer are verified:
   i. Stratum Corneum
   ii. Stratum Granulosum
   iii. Stratum Spinosum
   iv. Stratum Basale
   Evidence of the following cellular structures in the dermal layer are verified:
   v. Blood vessels, evidence of vasculature
   vi. Nerves
   vii. Various glands
   viii. Hair follicles
   ix. Collagen

It is therefore understood that multiple source animals, with an array of biological properties including, but not limited to, genome modification and/or other genetically engineered properties, can be utilized to reduce immunogenicity and/or immunological rejection (e.g., acute, hyperacute, and chronic rejections) in humans resulting from xenotransplantation. In certain aspects, the present disclosure can be used to reduce or avoid thrombotic microangiopathy by transplanting the biological product of the present disclosure into a human patient. In certain aspects, the present disclosure can be used to reduce or avoid glomerulopathy by transplanting the biological product of the present disclosure into a human patient. It will be further understood that the listing of source animals set forth herein is not limiting, and the present invention encompasses any other type of source animal with one or more modifications (genetic or otherwise) that serve(s) to reduce immunogenicity and/or immunological rejection, singularly or in combination.

By way of example, the present disclosure may be utilized to generate an array of proteins, cells, organs and/or tissues, through regenerative cell-therapy methods known in the art (e.g., through utilization of biological scaffolds), for xenotransplantation including, but not limited to, skin, kidneys, liver, brain, adrenal glands, anus, bladder, blood, blood vessels, bones, brain, brain, cartilage, ears, esophagus, eye, glands, gums, hair, heart, hypothalamus, intestines, large intestine, ligaments, lips, lungs, lymph, lymph nodes and lymph vessels, mammary glands, mouth, nails, nose, ovaries, oviducts, pancreas, penis, pharynx, pituitary, pylorus, rectum, salivary glands, seminal vesicles, skeletal muscles, skin, small intestine, smooth muscles, spinal cord, spleen, stomach, suprarenal capsule, teeth, tendons, testes, thymus gland, thyroid gland, tongue, tonsils, trachea, ureters, urethra, uterus, uterus, and vagina, areolar, blood, adenoid, bone, brown adipose, cancellous, cartaginous, cartilage, cavernous, chondroid, chromaffin, connective tissue, dartoic, elastic, epithelial, epithelium, fatty, fibrohyaline, fibrous, Gamgee, Gelatinous, Granulation, gut-associated lymphoid, Haller's vascular, hard hemopoietic, indifferent, interstitial, investing, islet, lymphatic, lymphoid, mesenchymal, mesonephric, mucous connective, multilocular adipose, muscle, myeloid, nasion soft, nephrogenic, nerve, nodal, osseous, osteogenic, osteoid, periapical, reticular, retiform, rubber, skeletal muscle, smooth muscle, and subcutaneous tissue.

In some aspects, the donor's genome is reprogrammed through specific combinations of precise, site-directed mutagenic substitutions or modifications whose design minimizes collateral genomic disruptions and has 5% or less net gain or net loss of total numbers of nucleotides and avoids genomic organizational disruption, and is non-transgenic, and that render the donor animal's cells, tissues, and organs tolerogenic when transplanted into a human without sacrificing the animal's immune function,

For example, a reprogrammed porcine donor genome may include endogenous exon and/or intron regions of the wild-type porcine donors' Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DQB and any combination thereof, that are disrupted, silenced or otherwise not functionally expressed on (95%) of extracellular surfaces achieved through specific combinations of precise, site-directed mutagenic substitutions or modifications;

In some aspects, the reprogrammed porcine donor genome comprises endogenous exon and/or intron regions of the wild-type porcine donor's PD-L1, CTLA-4, EPCR, TBM, TFPI, and/or MIC-2, and any combination thereof, that are humanized via reprogramming through specific combinations of precise, site-directed mutagenic substitutions or modifications with synthetic nucleotides from orthologous exons of a known human PD-L1, CTLA-4, EPCR, TBM, TFPI, and MIC-2 from the human captured reference sequence, designed from the human captured reference sequence and which minimizes collateral genomic disruptions and ideally results in no net gain or loss of total numbers of nucleotides and avoids genomic organizational disruption and that render the donor animal's cells, tissues, and organs tolerogenic when transplanted into a human without sacrificing the natural immune function of the B2M, PD-L1, CTLA-4, EPCR, TBM, TFPI, and MIC-2 proteins;

In some aspects, the reprogrammed porcine donor genome comprises endogenous exon and/or intron regions of the wild-type porcine donor's Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DQB, and any combination thereof, that are reprogrammed through specific combinations of precise, site-directed mutagenic substitutions or modifications with synthetic nucleotides from orthologous exons of a known human HLA-A, HLA-B, HLA-C, HLA-E, HLA-F, HLA-G, HLA-DRA, HLA-DRB, HLA-DQA, and/or HLA-DQB from the human captured reference sequence, designed from the human captured reference sequence and which minimizes collateral genomic disruptions and ideally results in no net gain or loss of total numbers of nucleotides and avoids genomic organizational disruption and that render the donor animal's cells, tissues, and organs tolerogenic when transplanted into a human without sacrificing the natural immune function of the SLA-1, SLA-2, SLA-3, SLA-6, SLA-7, SLA-8, SLA-DRA, SLA-DRB, SLA-DQA, and/or SLA-DQB proteins.

In some aspects, site-directed mutagenic substitutions are made in germ-line cells used to produce the non-human animal donor. In some aspects, the reprogrammed genome comprises at least one stop codon selected from TAA, TAG, and TGA, or a sequential combination of two or three of said stop codons. In some aspects, the reprogrammed genome comprises said at least one stop codon or said combination of two or three of said stop codons more than 70 base pairs downstream from the promoter of a gene or genes to be silenced such that the wild-type porcine donor gene lacks functional expression of said gene or genes.

In some aspects, the reprogrammed genome comprises reprogrammed nucleotides at one or more of a CTLA-4 promoter and a PD-L1 promoter, wherein the one or more of the CTLA-4 promoter and the PD-L1 promoter are reprogrammed to increase expression of one or both of reprogrammed CTLA-4 and reprogrammed PD-L1 compared to the wild-type donor animal's endogenous expression of CTLA-4 and PD-L1.

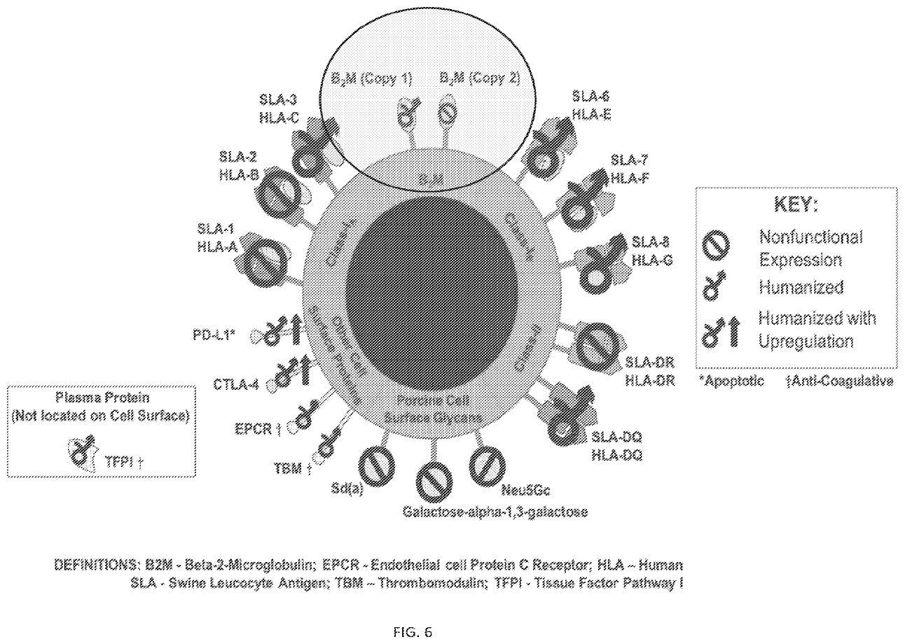

In some aspects, genetic modifications in a porcine cell line to insert the modifications listed in FIG. 6. In one aspect, the insertion of a genomic sequence construct in safe harbor locus ROSA26 of the porcine genome allows for gene expression without interrupting the function of essential endogenous genes. In some aspects, in addition to the genetic modifications listed in FIG. 6, the three predominant porcine donor cell surface glycans (Galactose-alpha-1,3-galactose (alpha-Gal), Neu5Gc, and/or Sda) are not expressed in order to reduce the hyperacute rejection phenomenon and the deleterious activation of antibody-mediated immune pathways, namely activation of the complement cascade. With this step, creation of an allogeneic-“like” cell with respect to non-MHC cell markers is grossly achieved.

In some aspects, the reprogramming with the plurality of synthesized nucleotides do not include replacement of nucleotides in codon regions that encode amino acids that are conserved between the wild-type donor animal's MHC sequence and the human captured reference sequence

In some aspects, the reprogrammed genome comprises site-directed mutagenic substitutions of nucleotides at the major histocompatibility complex of the wild-type donor animal with orthologous nucleotides from the human captured reference sequence.

In some aspects, the human captured reference sequence is a human patient capture sequence, a human population-specific human capture sequence, or an allele-group-specific human capture sequence.

In some aspects, the reprogrammed genome comprises site-directed mutagenic substitutions of nucleotides at exon regions of the wild-type donor animal's SLA-1 with nucleotides from an orthologous exon region of a HLA-A captured reference sequence.

In some aspects, the reprogrammed genome comprises site-directed mutagenic substitutions of nucleotides at exon regions of the wild-type donor animal's SLA-2 with nucleotides from an orthologous exon region of a HLA-B captured reference sequence.

In some aspects, the reprogrammed genome comprises site-directed mutagenic substitutions of nucleotides at exon regions of the wild-type donor animal's SLA-3 with nucleotides from an orthologous exon region of a HLA-C captured reference sequence.

In some aspects, the reprogrammed genome comprises site-directed mutagenic substitutions of nucleotides at exon regions of the wild-type donor animal's SLA-6 with nucleotides from an orthologous exon region of a HLA-E captured reference sequence.

In some aspects, the reprogrammed genome comprises site-directed mutagenic substitutions of nucleotides at exon regions of the wild-type donor animal's SLA-7 with nucleotides from an orthologous exon region of a HLA-F captured reference sequence.

In some aspects, the reprogrammed genome comprises site-directed mutagenic substitutions of nucleotides at exon regions of the wild-type donor animal's SLA-8 with nucleotides from an orthologous exon region of a HLA-G captured reference sequence.

In some aspects, the reprogrammed genome comprises site-directed mutagenic substitutions of nucleotides at exon regions of the wild-type donor animal's MHC class I chain-related 2 (MIC-2).

In some aspects, the reprogrammed genome lacks functional expression of SLA-1, SLA-2, SLA-DR, or a combination thereof.

In some aspects, the reprogrammed genome comprises site-directed mutagenic substitutions of nucleotides at exon regions of the wild-type donor animal's SLA-DQA from an orthologous exon region of a HLA-DQA1 captured reference sequence.

In some aspects, the reprogrammed genome comprises site-directed mutagenic substitutions of nucleotides at exon regions of the wild-type donor animal's SLA-DQB from an orthologous exon region of a HLA-DQB1 captured reference sequence.

In some aspects, the reprogrammed genome comprises site-directed mutagenic substitutions of nucleotides at exon regions of the wild-type donor animal's SLA-DRA and SLA-DRB1 with nucleotides from orthologous exon regions of HLA-DRAI and HLA-DRB 1 of the human captured reference sequence, or wherein the reprogrammed genome lacks functional expression of SLA-DRA and SLA-DRB1.

In some aspects, the reprogrammed genome comprises site-directed mutagenic substitutions of nucleotides at exon regions of the wild-type donor animal's SLA-DQA and SLA-DQB1 with nucleotides from orthologous exon regions of HLA-DQA1 and HLA-DQB1 of the human captured reference sequence.

In some aspects, the site-directed mutagenic substitutions of nucleotides are at codons that are not conserved between the wild-type donor animal's genome and the known human sequence.

As shown in a comparison of the amino acid sequences of Sus scrofa B2M to hB2M, 87/120 amino acids are the same so human B2M protein shares 70% sequential identity with pB2M. Copies 1 and 2 of porcine have identical exons to each other.

Porcine wild type B2M:
(SEQ ID NO: 6)
MAPLVALVLLGLLSLSGLDAVARPPKVQVYSRHPAENGKPNYLNCYVSG
FHPPQIEIDLLKNGEKMNAEQSDLSFSKDWSFYLLVHTEFTPNAVDQYS
CRVKHVTLDKPKIVKWDRDH*
Human Wild type B2M:
(SEQ ID NO: 5)
MSRSVALAVLALLSLSGLEAIQRTPKIQVYSRHPAENGKSNFLNCYVSG
FHPSDIEVDLLKNGERIEKVEHSDLSFSKDWSFYLLYYTEFTPTEKDEY
ACRVNHVTLSQPKIVKWDRDM*
Porcine Edited copy 1 (A2 line):
(SEQ ID NO: 41)
RPPKVQVYSRHPAENGKPNYLNCYVSGFHPPQIEIDLLKNGEKMNAEQS
DLSFSKDWSFYLLVHTEFTPNAVDQYSCRVTLDKPKIVKW
Porcine edited copy 2 (A2 line):
(SEQ ID NO: 42)
RPPKVQVYSRHPAENGKPNYLNCYVSGFHPPQIEIDLLKTGRR*TRSSQ
TCLSARTGLSTFWSTLSSLLTLWISIAAAWKHVTLDKPKIVKW

A2 is one of the two clones that were made with a modification of both of the B2M genes and confirmed to not express B2M on the cell surface.

### Examples

Molecular analysis, Western Blot, is performed for the β2M protein in the cytosol of a porcine cell to verify that copy 1 and copy 2 of the β2M gene are inactivated from the porcine genome.

Phenotype analysis is performed for lack of detection of MHC class I on the cell surface of a porcine cell to verify that copy 1 and copy 2 of the β2M gene are inactivated from the porcine genome.

An enzyme-linked immunosorbent assay (ELISA) is used to measure release of free hβ2M and the absence of pβ2M into the media of cells in culture after reprogramming of the porcine cell using CRISPR-Cas9 for site-directed mutagenic substitutions to mediate rapid and scarless exchange of pβ2M gene to hβ2M gene.

Intracellular cytokine staining measured by flow cytometry is performed to detect cytokines and to link their expression to the phenotype of porcine cells containing the humanized β2M gene.

### Designing Species Specific Monoclonal Antibodies

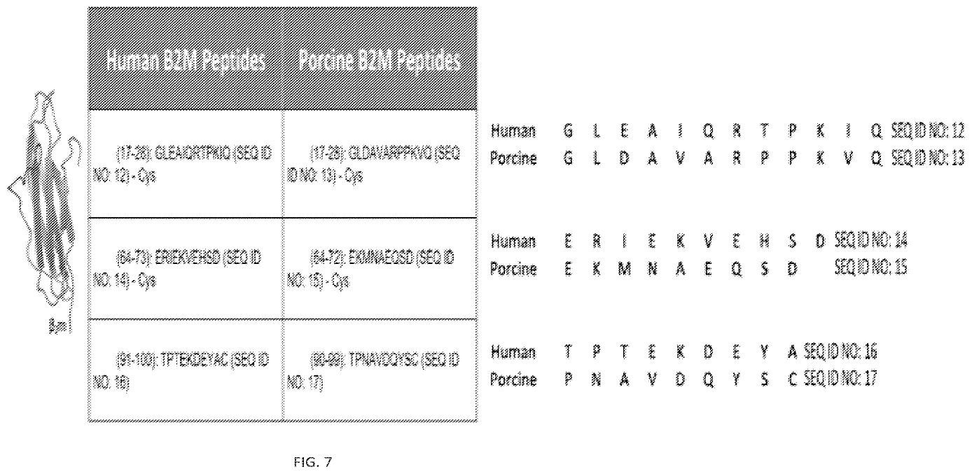

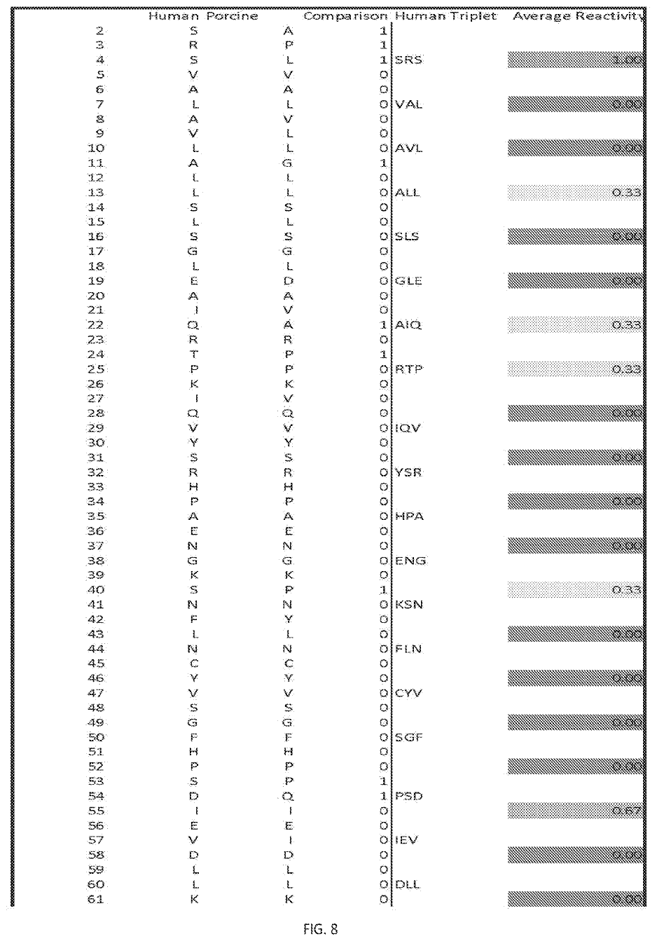

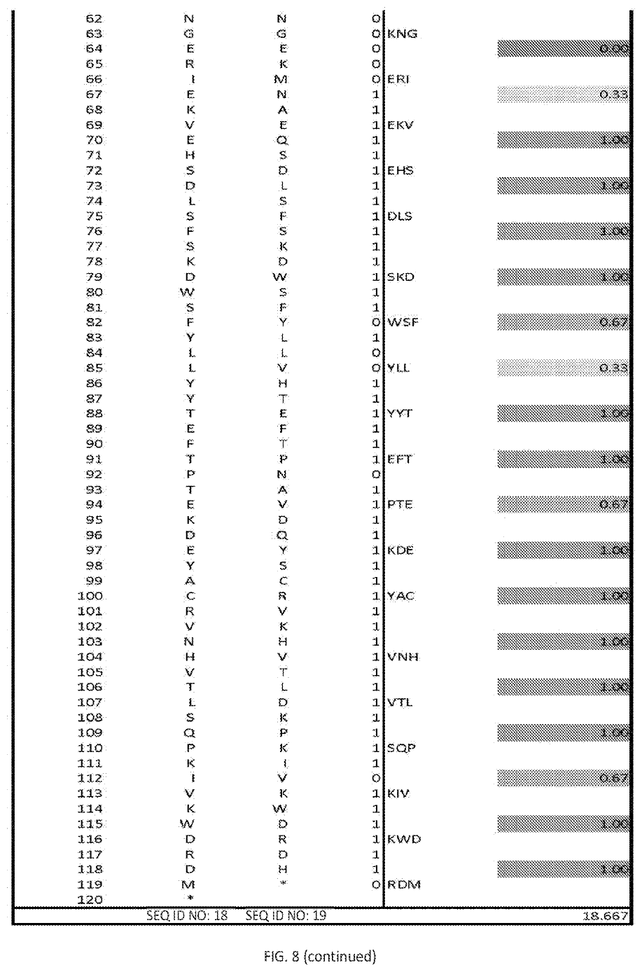

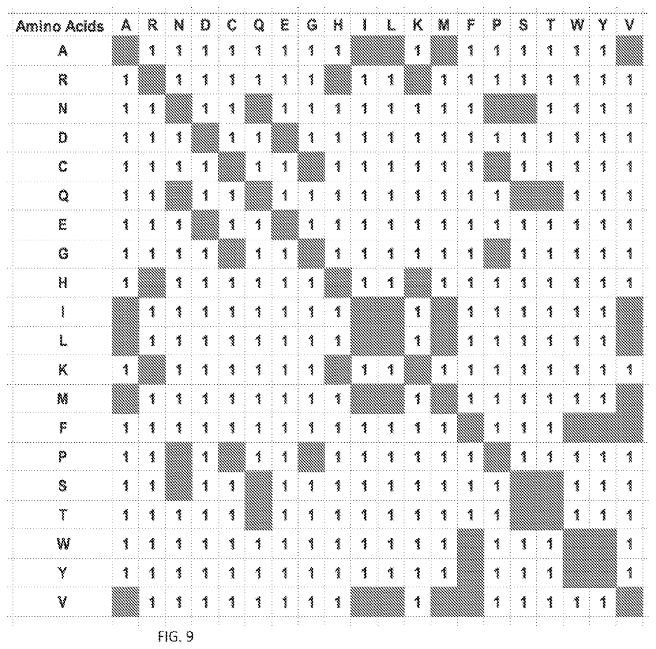

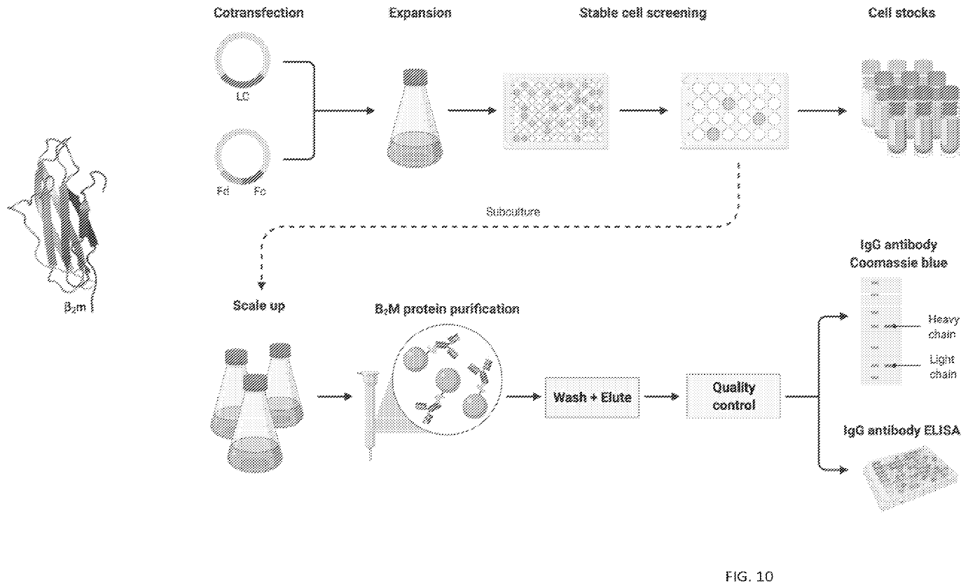

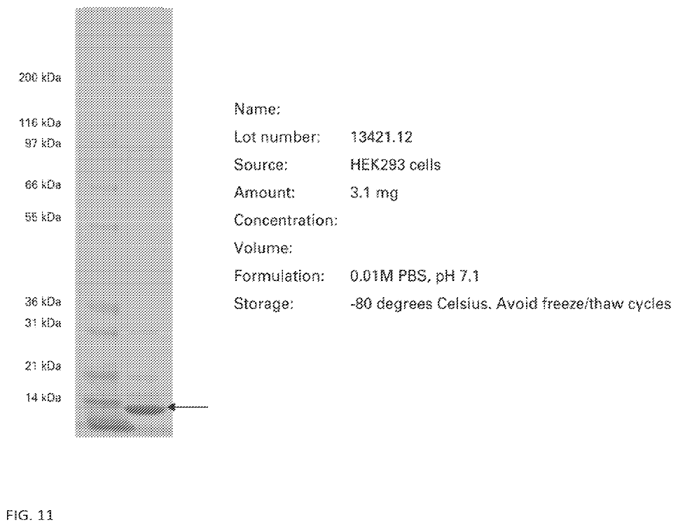

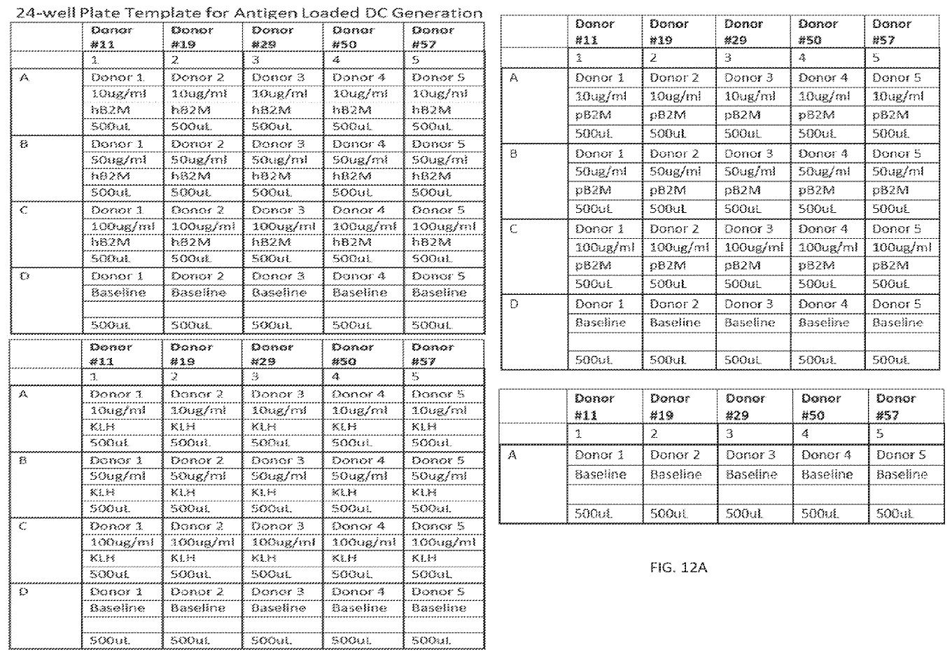

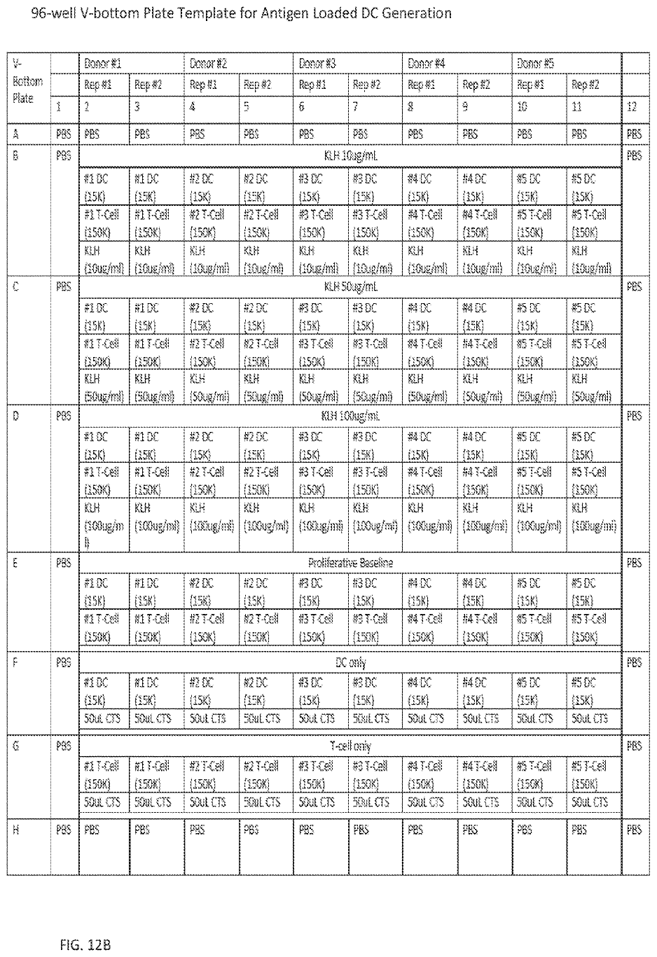

Off the shelf antibodies today cannot determine the difference between hβ2M and pβ2M. Therefore, custom, species-specific antibodies are required. Each peptide avoids continuously conserved stretches of greater than 4-6 amino acids that may constitute conserved epitopes and allow for specific binding with the human or porcine binding proteins as shown in the key areas of antigenicity shown in FIG. 7. As shown in FIG. 8 and FIG. 9, analysis was performed to assess whether a B2m mismatch within a CREG group may result in a better outcome than a mismatch outside CREG groups. Validated outcomes data were analyzed against known amino acid characteristics. Each region also looks very good in terms of predicted immunogenicity/exposure. Antibodies developed against these peptides would only require a single affinity purification against the peptide immunogen to ensure specificity against each of the target proteins. The production process to design pβ2M Recombinant Monoclonal Antibodies for Antigenicity Testing is shown using dynamic BioRender assets as demonstrated in FIG. 10. As shown in FIG. 11, the porcine recombinant B2M protein was produced.

### Selection and Characterization of Pilot Porcine Cell Line

Primary macrophages and other antigen presenting cells (APC) are useful for studying immune response, however, the long-term use of primary cells is limited by the cells' short life span. In addition, primary cells can only be genetically engineered and evaluated one time before the cells reach senescence. In the pig model, investigators frequently have used porcine aortic endothelial cells (PAECs) for these type of studies. An immortalized cell line that has the desired characteristics (expression of MHC Class I and II molecules and CD80/86) of a macrophage or representative APC would be ideal to conduct multiple modifications of the genome and address impact on immunological reactivity using the same genetic background. The ability to generate a viable immortalized pig cell line has been limited to fibroblasts and epithelial cell lines which are not relevant for the study of the immune response in xenotransplantation.

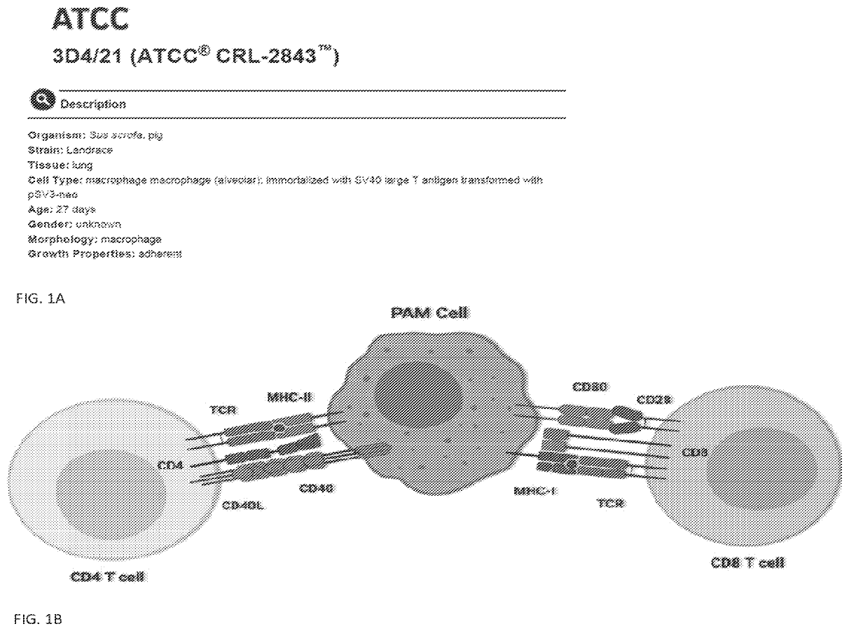

An immortalized porcine alveolar macrophage (PAM) line was developed from Landrace strain of pig [Weingartl 2002] and is commercially available through ATCC® [3D4/21, ATCC CRL-2843TM]. Another such cell line is 3D4/2 (ATCC® CRL-2845TM). The surface characterization of the PAM cell (3D4/21) is demonstrated in FIG. 1A-1B. The cell line showed some percentage of non-specific esterase and phagocytosis which was dependent upon conditions of the medium. Cells could be grown as anchorage dependent or in colonies under serum free conditions. Myeloid/monocyte markers (e.g., CD14) were detected. Desired characteristics of an immortalized cell line was MHC Class I and II. MHC Class I was shown to be broadly expressed on all cells, however, MHC Class II, DR and DQ, expression of 3D4/21 cells was initially reported as being low, 18% and 4%. PAEC have been shown to be activated and DR expression could be upregulated with exposure to IFN-gamma. 3D4/21 cells were exposed to IFN-gamma and Class II expression increased DR: 29.68% to 42.27% and DQ: 2.28% to 57.36% after 24 hours of exposure to IFN-gamma. In addition, CD80/86 are expressed on the cell surface, these glycoproteins are essential for the second signal of T-cell activation and proliferation. PAM cells, 34D/21, have the desired characteristics of a porcine APC in which genetic changes in genes associated with the MHC can be documented using an immortalized cell line and the resulting changes in the phenotype can be assessed using flow cytometry to address expression or lack of expression of the glycoproteins of interest and cellular immune responses, Mixed Lymphocyte Response (MLR).

To test for cellular immune response, a one-way MLR is set up in which one set of cells is identified as the stimulator cells, these are donor cells or unmodified or modified PAM cells, and the other set of cells is the responder cells, these are cells from the recipient (these could be from recipient's who share a similar expression of MHC molecules are the modified PAM cells. The stimulator cells are treated with an agent to prevent the cells from proliferating, and this could be either radiation or incubation with mitomycin C which covalently crosslinks DNA, inhibiting DNA synthesis and cell proliferation. Hence, the stimulator cells do not proliferate in culture however, the responder cells proliferate in response to interaction at the MHC Class I and II and it is this proliferation that is measured in an MLR. A cell culture containing both stimulator and responder cells is prepared and incubated for 5-7 days, and proliferation/activation is measured. Proliferation can be measured by the amount of radioactive thymidine [3HTdr] or BrdU [analog of thymidine] that is incorporated into the DNA upon proliferation at the end of 5 or 7 days.

Combinations of the MLR. Responder cells can be either PBMC, CD4+ T-cells, CD8+ T-cells or other subpopulations of T-cells. PBMC represent all the immune cells that are present in the recipient and the measured response reflects the ability of the responders to mount an immune response to the stimulator cells, [unmodified or modified PAM cells]. The measured proliferation consists of both CD4+ and CD8+ T-cells which interact with MHC Class II and I, respectively. Using only CD4+ T-cells against the unmodified or modified PAM cells is to measure the response to MHC Class 11 glycoproteins, DR and DQ. To observe a specific response to DQ, human antigen presenting cells (APCs) are absent from the culture such that the cellular response is not the result of pig antigens presented by the APCs. In parallel, responder CD8+ T-cells will be used to assess an immune response to MHC Class I glycoproteins, SLA 1 AND 2. This type of analysis removes the contribution to the immune response from responder APCs as found in PBMC. Comparative data will demonstrate the contribution of these respective glycoproteins to the immune response of the genetically defined responder and reflects on the genetic modifications made to the PAM cells.

Flow cytometry, phenotypic analysis of the genetically engineered PAM cells. The cell phenotype of genetically engineered cells, e.g., cells from a genetically engineered animal or cells made ex vivo, are analyzed to measure the changes in expression of the glycoproteins encoded by the genes that were modified. Cells are incubated with an antibody with a fluorescent label that binds to the glycoprotein of interest and labeled cells are analyzed using flow cytometry. The analysis has been performed on unmodified PAM cells to identify the expression of MHC Class I, Class II (DR and DQ) and CD80/86. Changes in modified PAM cells will be referenced to this database. Flow cytometry will also be used to characterize the expression of glycoproteins encoded by genes for SLA 3, 6, 7, and 8 as the genes in the PAM cells are modified with recipient specific sequences related to HLA C, E, F, and G.

In addition, this type of analysis is also used to ensure the glycoprotein encoded by a gene that is knock-out is not expressed. This technique can also be used to sort out genetically engineered cells from a pool of cells with mixed phenotypes.

Complement Dependent Cytotoxicity (CDC) assays may be performed to determine if anti-HLA antibodies recognize the cells from the biological product of the present disclosure. Assay plates prepared by adding a specific human serum containing previously characterized anti-HLA antibodies (or control serum) can be used. IFN-γ treated donor cells are resuspended and added to the assay plates, incubated with a source of complement, e.g., rabbit serum. After at least 1 hour of incubation at room temperature, acridine orange/ethidium bromide solution is added. Percent cytotoxicity is determined by counting dead and live cells visualized on a fluorescent microscope, subtracting spontaneous lysis values obtained in the absence of anti-HLA antibodies, and scoring with a scale.

NK cell reactivity, modulation to decrease cytotoxicity. Potential mechanisms of activation, recognition, and elimination of target cells by NK cells, alone or in combination, induce the release of the content of their lytic granules (perforin, granzyme, and cytolysin). As an example, NK cells recognize the lack of self-major histocompatibility complex (MHC) Class I molecules on target cells by inhibitory NK cell receptors leading to direct NK cytotoxicity. This is the case for xenotransplantation. NK cells are regulated by HLA C that is recognized by inhibitory NK cell inhibitory killer cell immunoglobulin-like receptors (KIRs), KIR2DL2/2DL3, KIR2DL1, and KIR3DL1. NK cells inhibitory receptor, immunoglobulin-like transcript 2 (ILT2) interacts with MHC Class I and CD94-NKG2A recognizing HLA-E. HLA F and G have similar roles on the trophoblast. The cytolytic activity of recipient NK cells to an unmodified PAM cell can be measured in vitro in which human NK cells are added to an adherent monolayer of unmodified PAM cells and cultured for 4 hours. Cell lysis is measured by release of radioactive Cr51, or a chromophore measured by flow cytometry. PAM cells with modified porcine genes can be assessed using this cytotoxicity assay.

For knock in cells, the desired sequences are knocked into the cell genome through insertion of genomic material using, e.g., homology-directed repair (HDR). To optimize expression of Class II molecules, the cells are incubated in porcine interferon gamma (IFN-γ) for 72 hours which stimulates expression. Expression is then measured by flow cytometry using target specific antibodies. Flow cytometry may include anti-HLA-C, HLA-E, HLA-G, or other HLA antibodies, or pan anti-HLA Class I or Class II antibodies. According to the present disclosure, cell surface HLA expression after knock-in is confirmed.

A study was conducted identify the impact of the stimulation by IFN-7 and IFN-7+LPS on the phenotype of the porcine alveolar macrophages (PAM) purchased from ATCC® (3D4/21 cells cat #CRL-2843™) by flow cytometry.

PAM cells were thawed in RPMI-1640/10% FBS and cultured for two days in three different culture plates. On Day 3, for macrophage activation culture medium was replaced with RPMI-1640/20% FBS medium containing 100 ng/mL IFN-γ (Plate 1) and 100 ng/mL IFN-γ plus 10 ng/mL LPS (Plate 2). Untreated cells in RPMI-1640/20% FBS were used as control (Plate 3). Following 24 hours incubation, adherent cells were detached from the plate using TrypLE treatment. Cells were resuspended in FACS buffer (1×PBS pH=7.4, 2 mM EDTA, 0.5% BSA). Cell count and viability were determined by trypan blue exclusion method. A total of 1×105 cells were stained with mouse anti pig SLA Class I, SLA Class II DR, SLA Class II DQ antibodies for 30 min and APC-conjugated CD152(CTLA-4)-mulg fusion protein (binds to porcine CD80/CD86) for 45 min at 4° C. Cells were washed two times using FACS buffer and antibody-stained cells resuspended in 100 L FACS buffer containing anti mouse APC-conjugated polyclonal IgG secondary antibody. Followed by incubation for 30 min at 4° C. Cells were washed two times using FACS buffer. All cells were resuspended in 200 LL FACS buffer. Samples were acquired in Novacyte flow cytometry and data was analyzed using NovoExpress.

Analysis procedure is based on NovoExpress flow cytometry analysis software. Any equivalent software can be used for the data analysis. Depending on the software used analysis presentation maybe slightly different. Gates maybe named differently and % values might be slightly different.

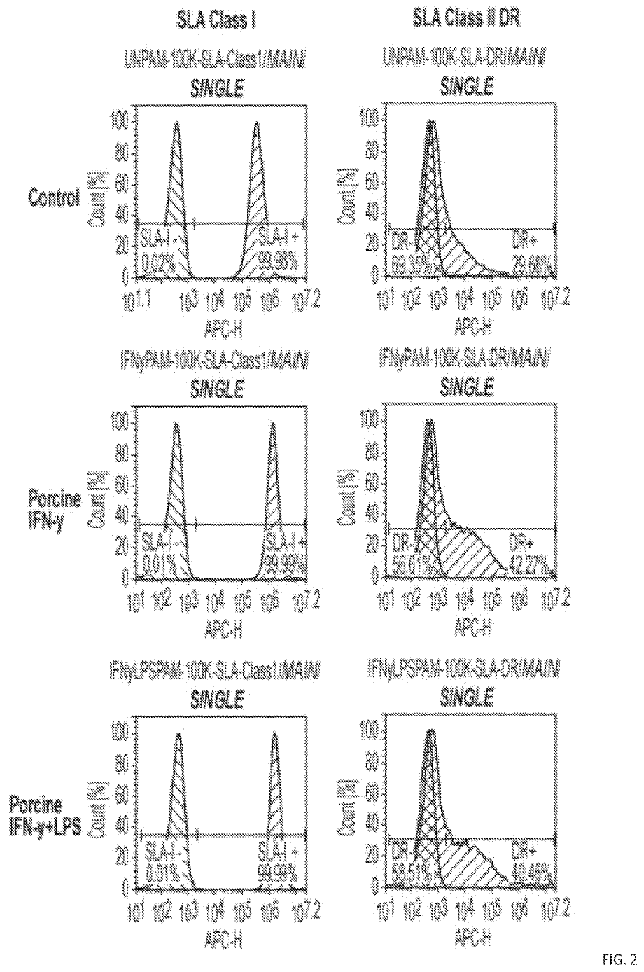

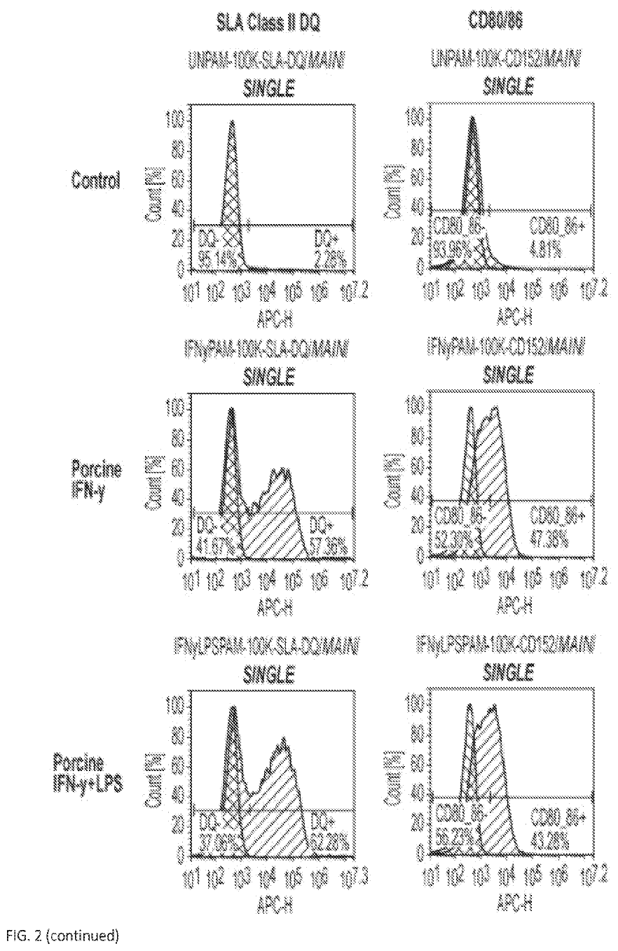

As shown in FIG. 2, untreated PAM cells result 99.98%, 29.68%, and 2.28% SLA Class I, SLA Class II DR and DQ molecules expression respectively. These cells were 4.81% CD80/86+. 24 hours of culturing cells in the presence of IFN-γ increased all SLA molecule expression (99.99/6 SLA Class I+ with increased median fluorescence intensity, 42.27% DR+, 57.36% DQ+) and CD80/86 levels (47.38%). IFN-γ containing cells with LPS resulted similar levels of SLA molecules and CD80/86 expression compared to cells only treated with IFN-γ.

PAM cells were treated with porcine IFN-γ for 24 hours and stained with primary mAbs and fluorescein conjugated secondary antibody and APC conjugated CD152 which has a high affinity for co-stimulatory molecules CD80 (B7-1) and CD86 (B7-2). Upon treatment with IFN-γ, the cells displayed increased SLA and CD80/86 costimulatory molecules expression compared to unstimulated PAM cells. While unstimulated cells were 99.98% SLA Class I+, 29.68% DR+2.28 DQ+ and 4.81% CD80/86+, IFN-γ stimulated cells were 99.99% SLA Class I+, 42.27% DR+, 57.36% DQ+, 47.38% CD80/86+. IFN-γ containing cells with LPS resulted similar levels of SLA molecules and CD80/86 expression compared to cells only treated with IFN-γ.

In basal conditions, macrophages express low levels of SLA Class II and CD80/86 costimulatory molecules. IFN-γ and IFN-7-LPS treatment for 24 hours induces the expression of SLA Class II and CD80/86 costimulatory molecules as well as SLA Class I molecules. Extended incubations would perhaps increase the expression of these molecules further.

### Knockout of Porcine-β2M Protein

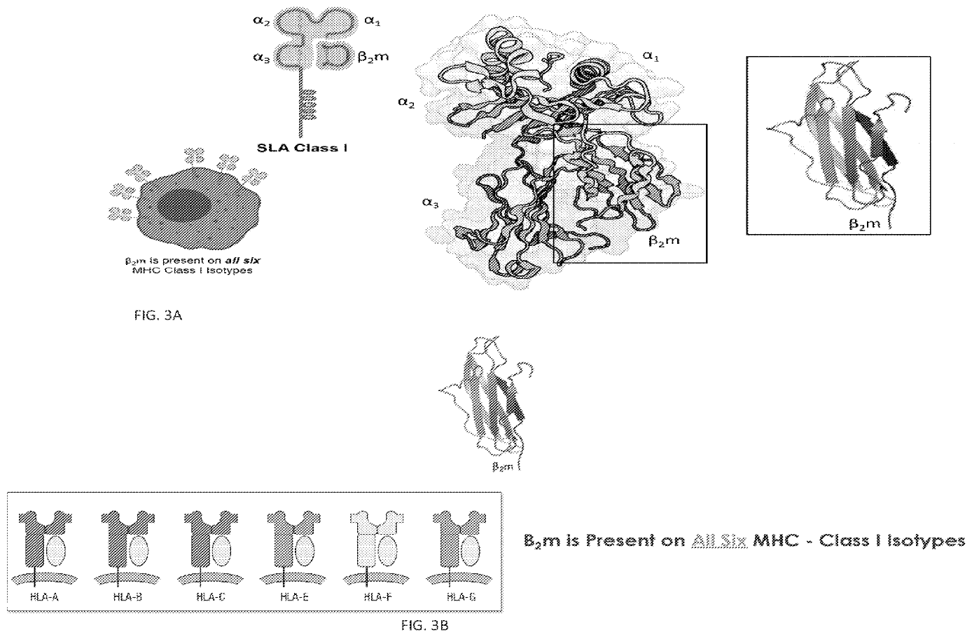

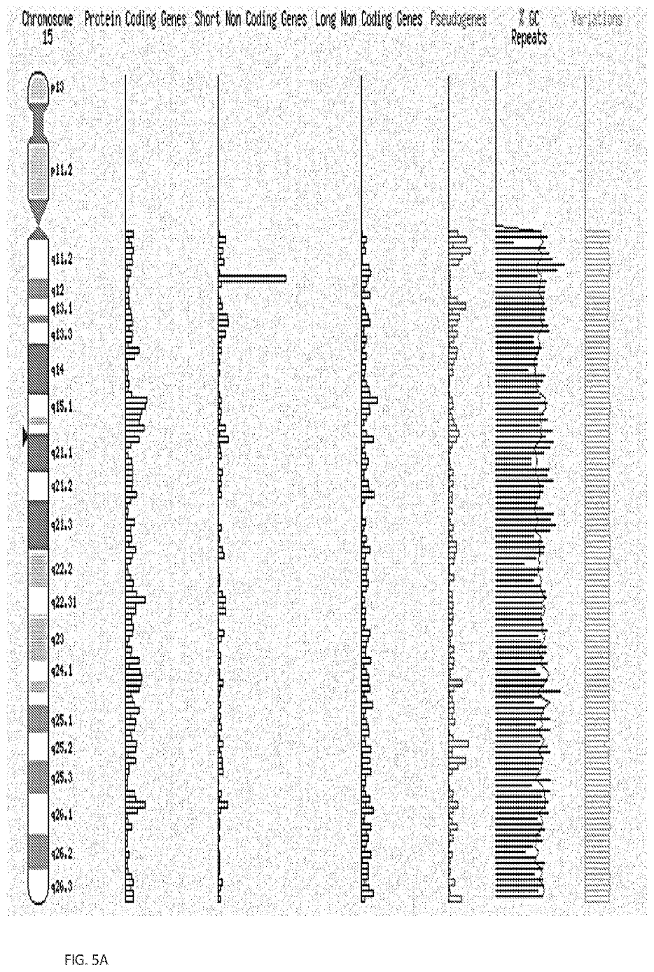

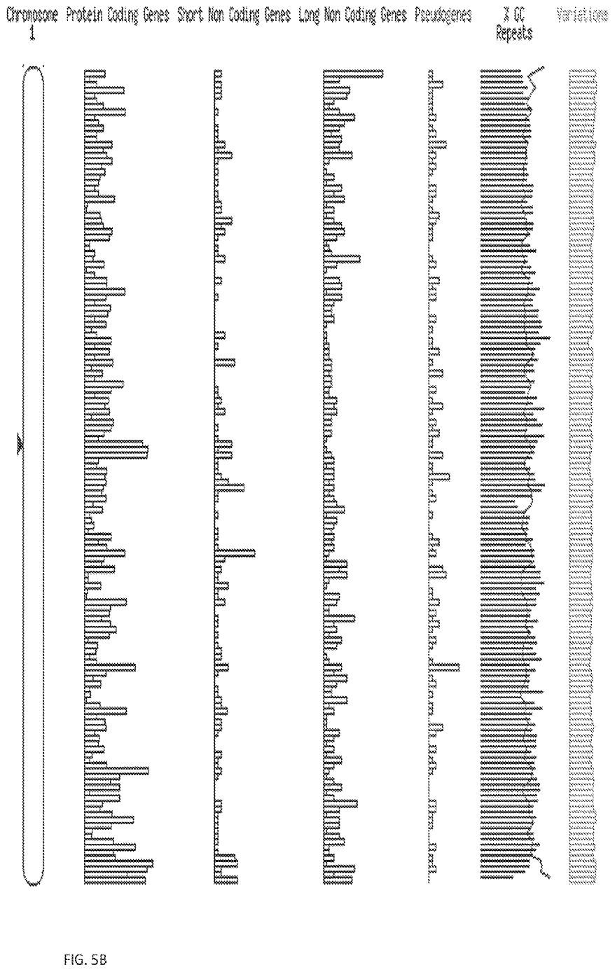

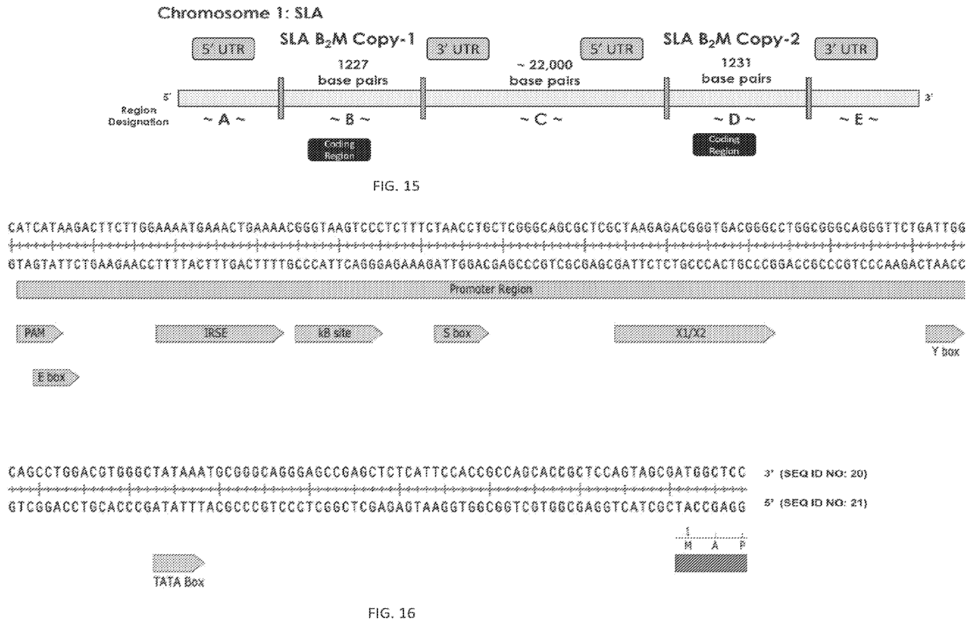

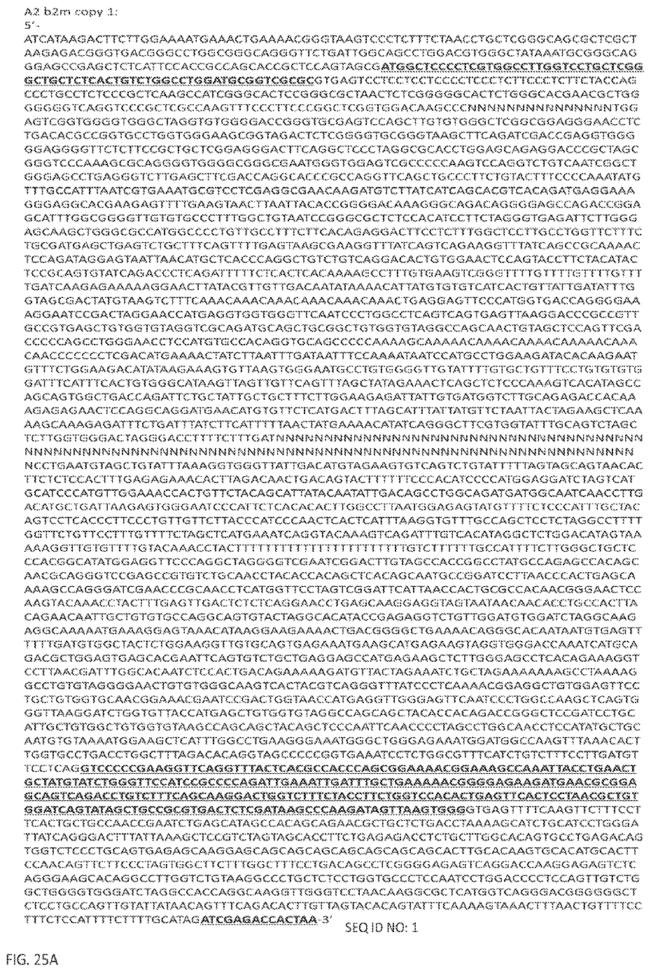

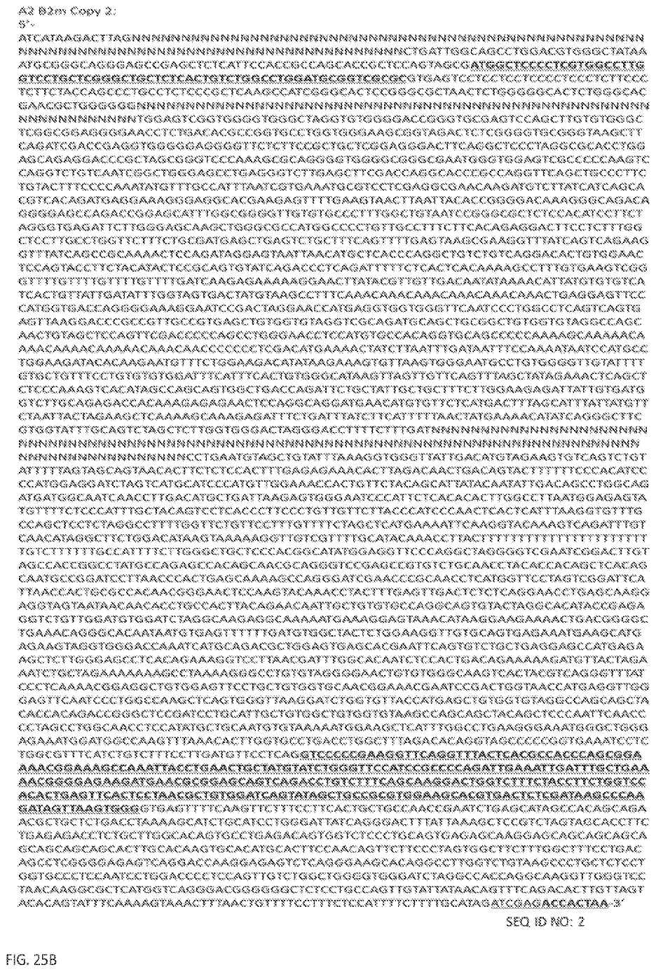

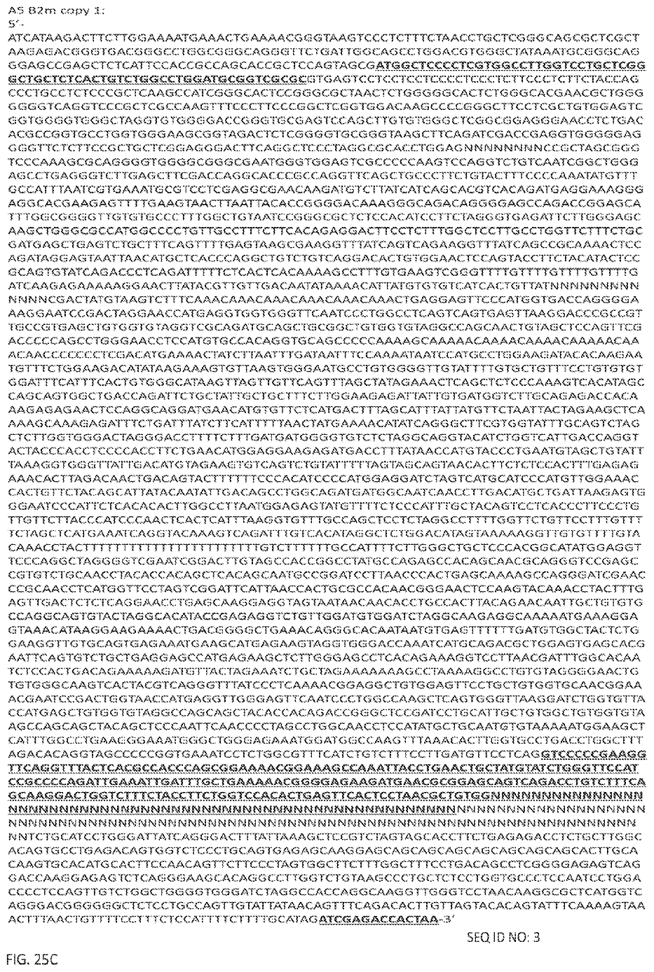

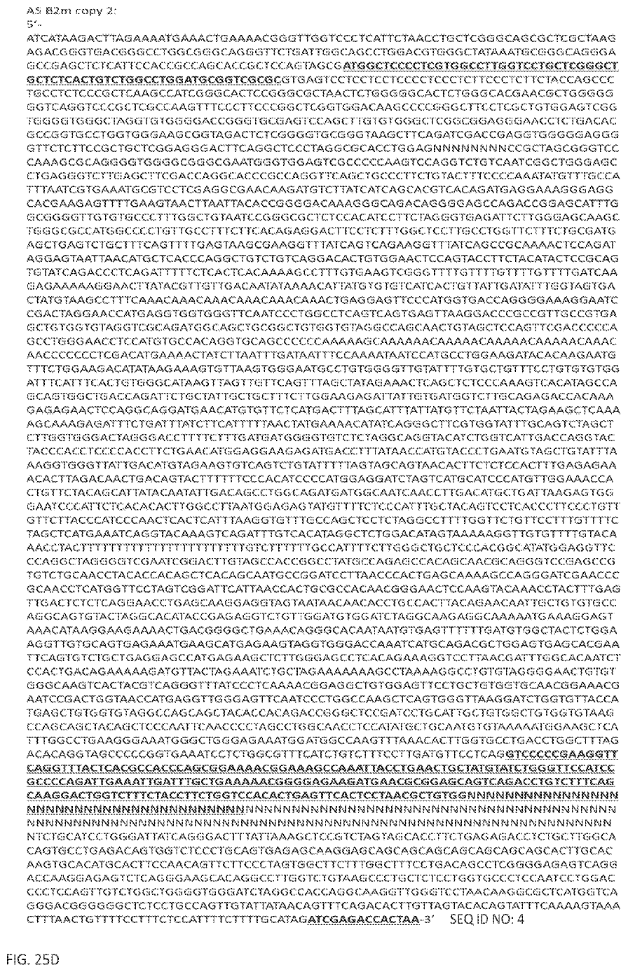

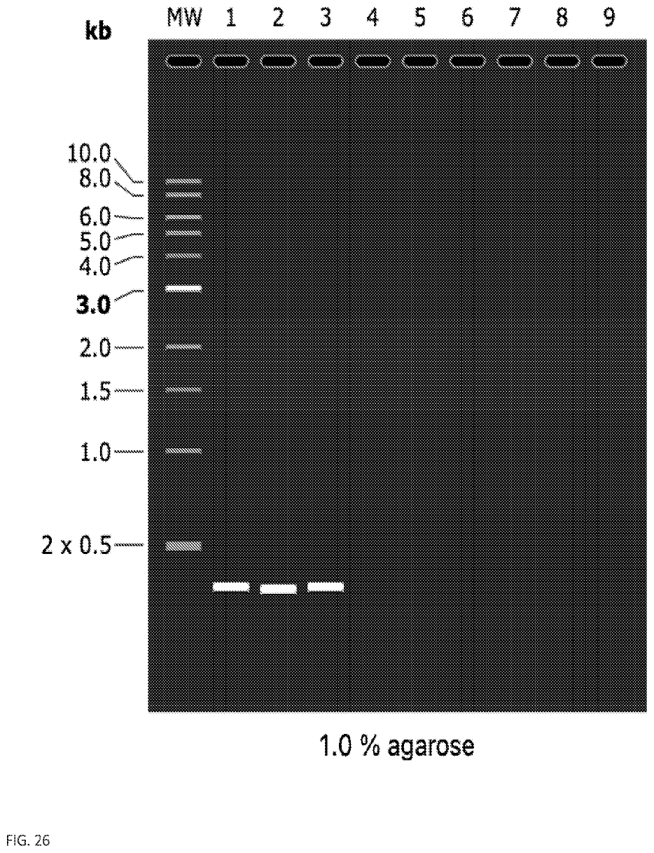

In contrast to humans, the porcine-β2M protein (pβ2M) gene is approximately 45.5 kb, due to an identical duplication of the β2M gene (copy 1 and copy 2) on the chromosome 1, separated by a unique intronic region. Comparison of porcine β2M on Chromosome 1 to the human β2M gene on Chromosome 15 is illustrated in FIG. 5A. Genomic organization of the porcine Beta-2-Microglobulin (B2M), a light beta-chain located on Chromosome 1 is illustrated in FIG. 5B. As further demonstrated in FIGS. 3B and 15, B2M is present on all six MHC Class I Isotypes in both the human and porcine; however, porcine have a functional duplication of the β2M gene (copy 1 and copy 2). The 2D and 3D protein structures of the MHC-antigen peptide complex for MHC Class I are illustrated in FIG. 3A, including Beta-2-Microglobulin ($2M), a light beta-chain, is present on all six MHC Class I Isotypes. A comprehensive sequences of copy 1 and 2 of β2M gene of A2 and A5 clones in which the pβ2M copy 1 and copy 2 nucleotide sequence has been removed and did not express β2M is demonstrated in FIGS. 25A-25D.

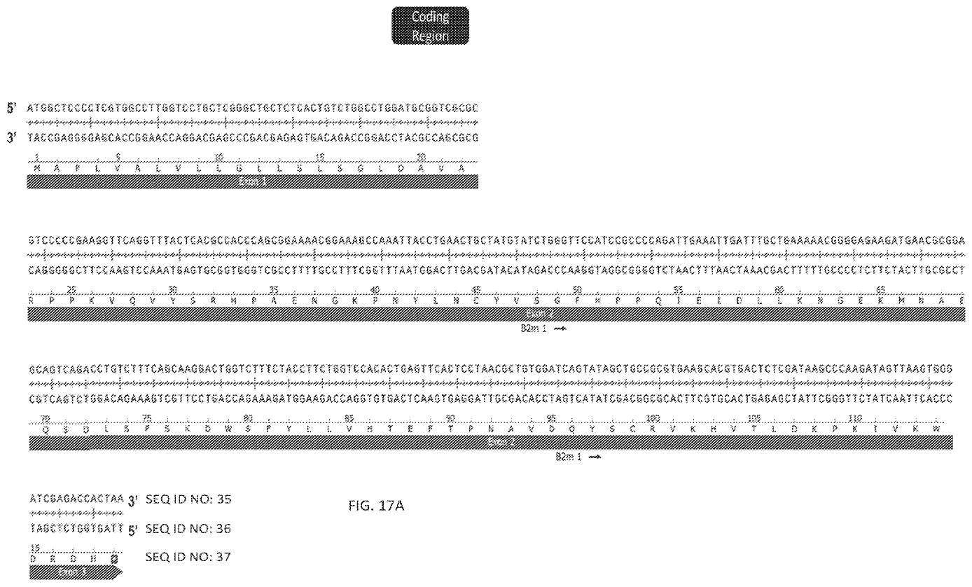

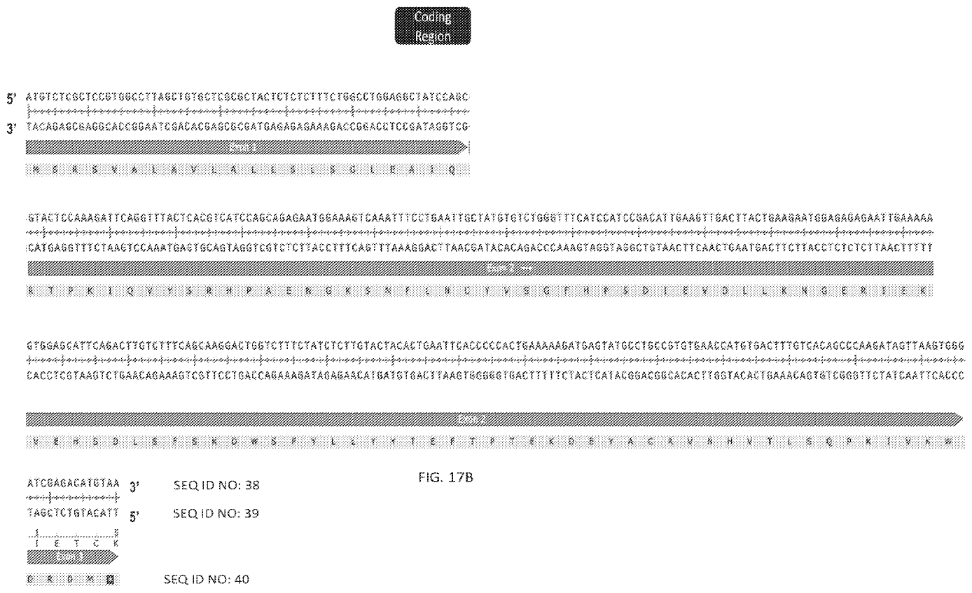

FIG. 15 shows Genomic organization of the porcine Beta-2-Microglobulin (B2M), a light beta-chain located on Chromosome 1. B2M is present on all six MHC Class I Isotypes in both the human and swine; however, swine have a functional duplication of the B2M gene (copy 1 and copy 2). Humans possess only a singular copy of the B2M gene. Note that the scale is approximate. FIG. 16 shows schematic of porcine promoter region.

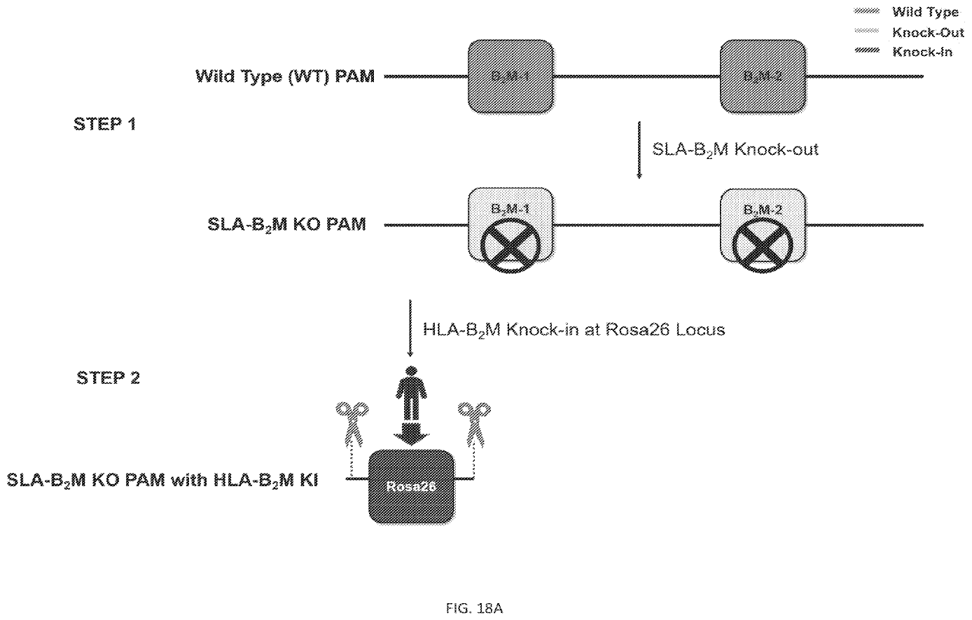

FIG. 17A shows a schematic of a porcine B2M coding region. FIG. 17B shows a schematic of a human B2M coding region.

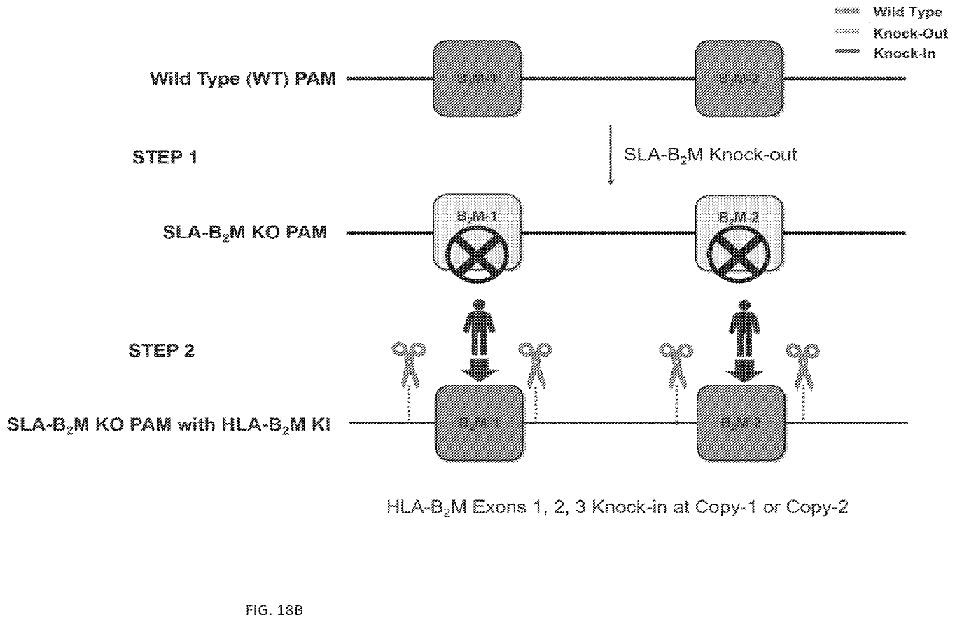

As shown in FIG. 18A, the steps for gene-editing include: (1) A knockout of the porcine B2M Copy 1 and Copy 2 using a large fragment deletion at exon 2; (2) Insertion of B2M from a designated human donor was placed into the Rosa26 safe harbor site. Light grey is used to indicate a knockout or fragdel has occurred, blue is representative of a human donor knock-in of the gene.

As shown in FIG. 18B, the steps for gene-editing include: (1) A knockout of the porcine B2M Copy 1 and Copy 2 using a large fragment deletion at exon 2; (2) Insertion of B2M from a designated human donor was placed into either Copy 1 or Copy 2. Light grey is used to indicate a knockout or fragdel has occurred, blue is representative of a human donor knock-in of the gene.

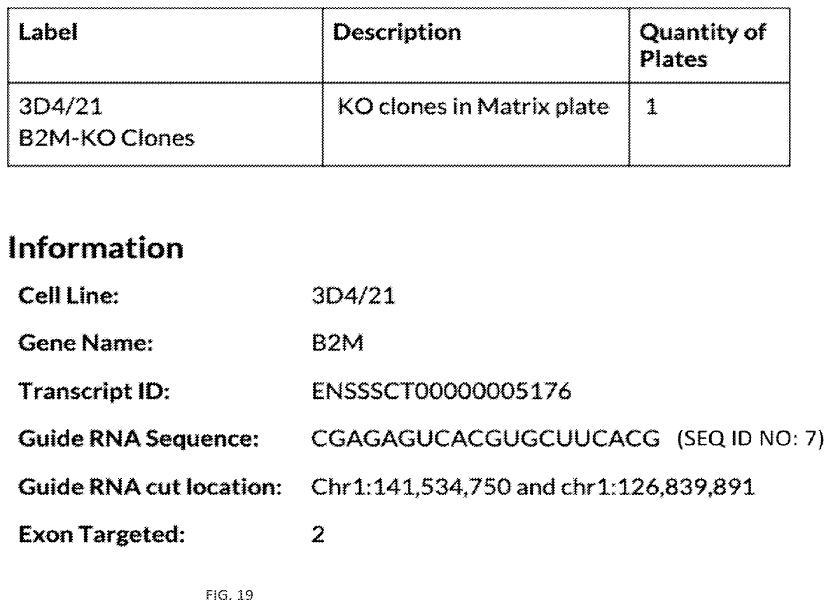

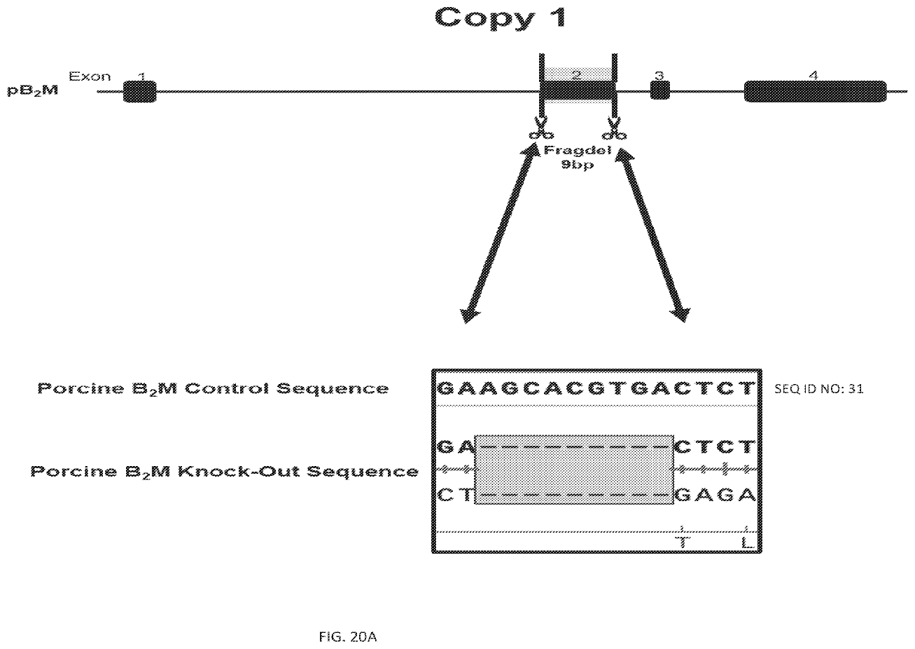

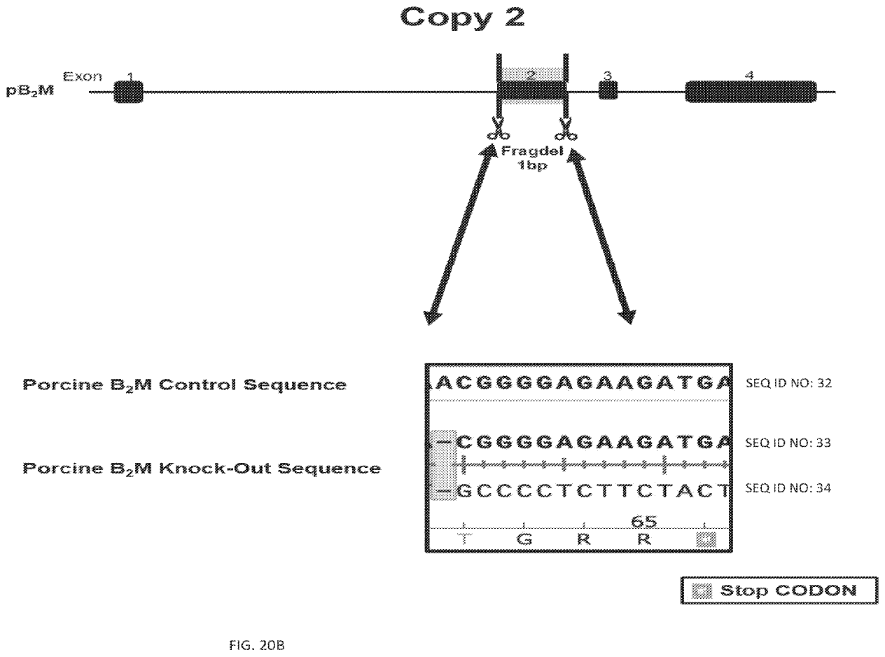

FIG. 19 shows that the specific guide RNAs are complexed together with the sp Cas9 to form a ribonucleoprotein (RNP). RNPs are then delivered to the cells via the optimized electroporation setting identified using a 200 point.

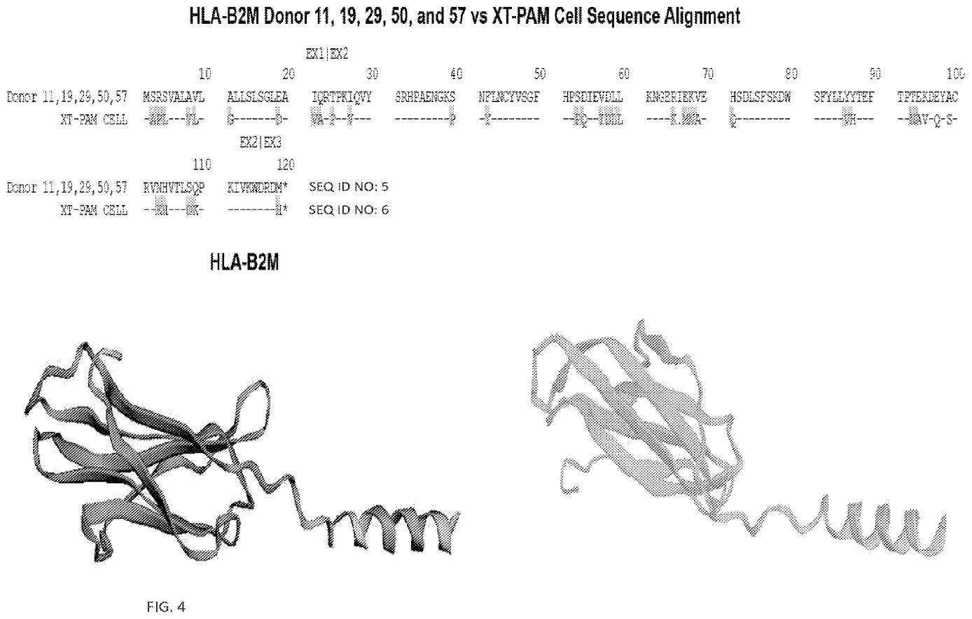

A comparison of amino acid sequences of Beta-2-Microglobulin among pigs and humans is provided in FIG. 4. Amino acid sequences were compared throughout the entire coding region to evaluate sequence conservation. Identical residues are indicated by dashes. Exon regions are marked above the sequences and the numbers above the sequence indicate the number of amino acids starting from exon 1 of the domain.

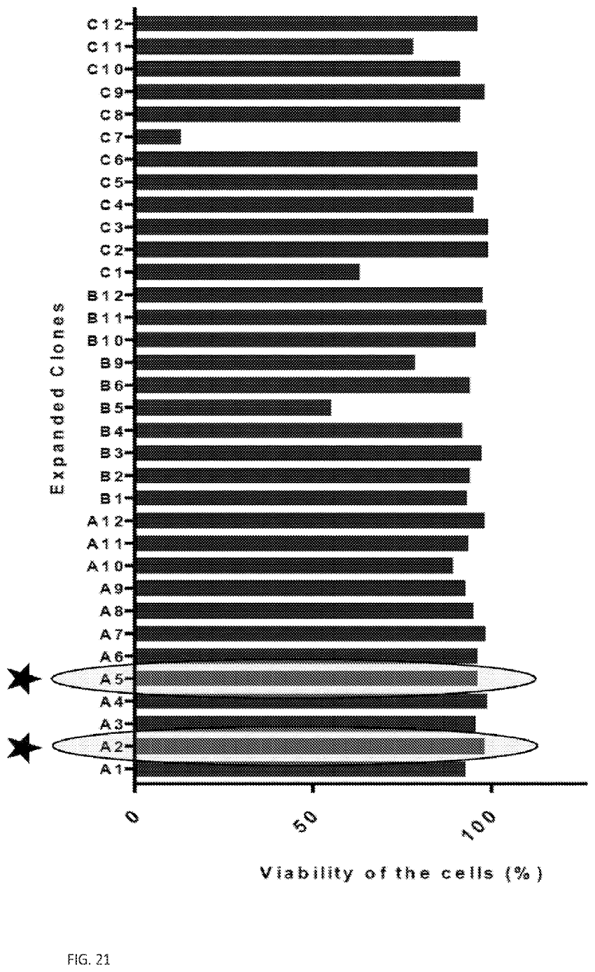

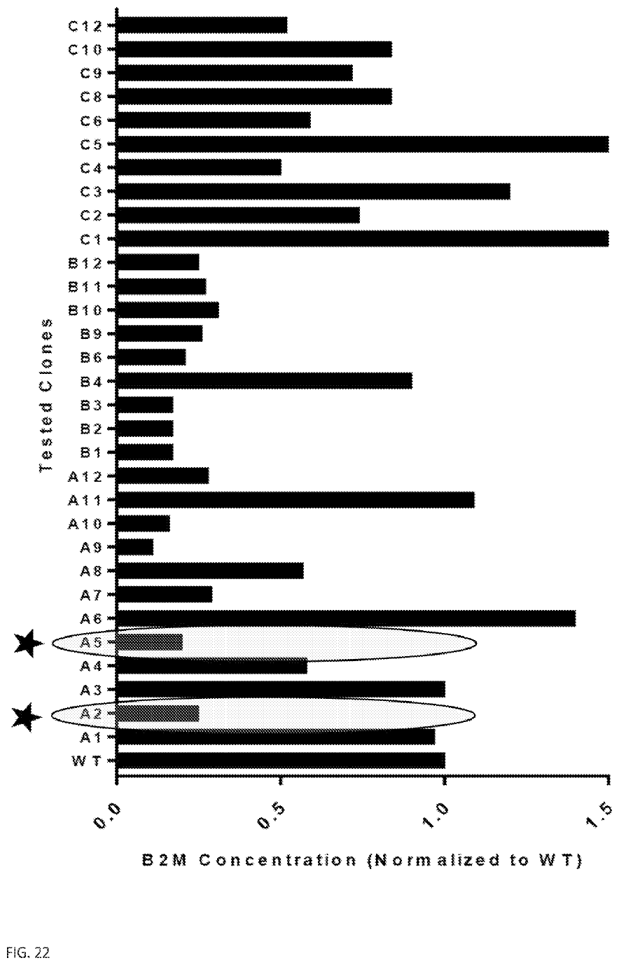

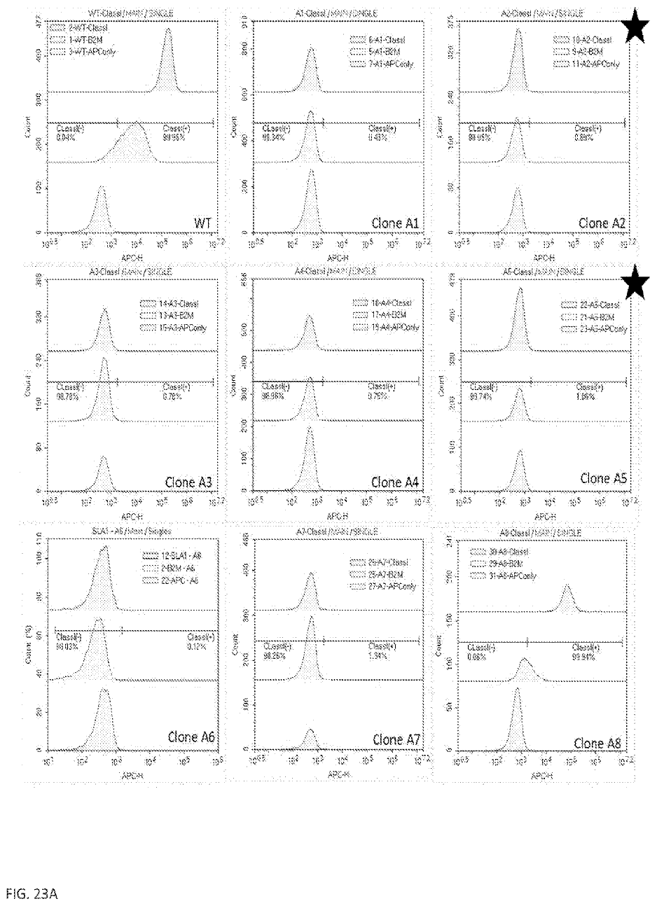

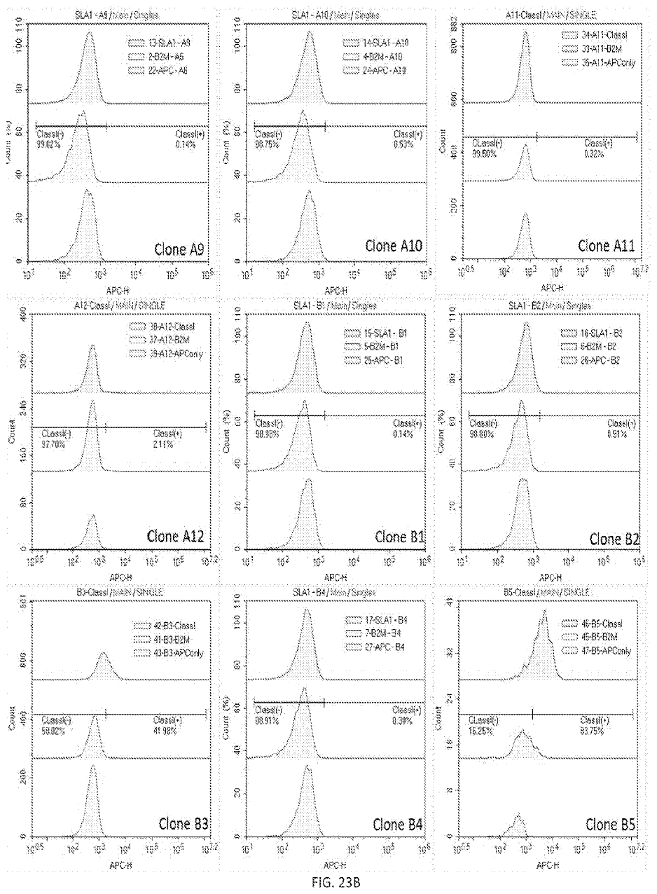

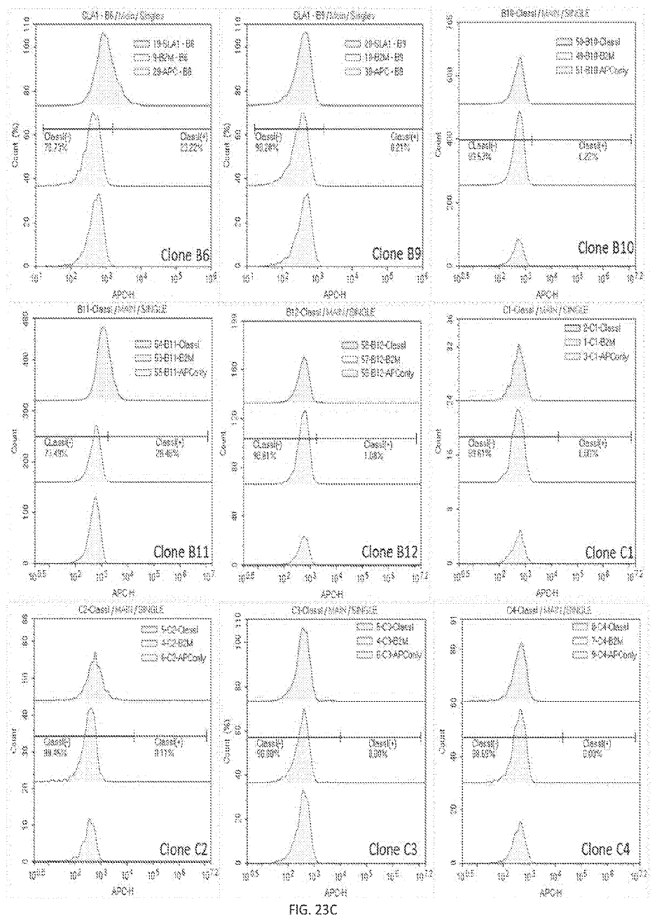

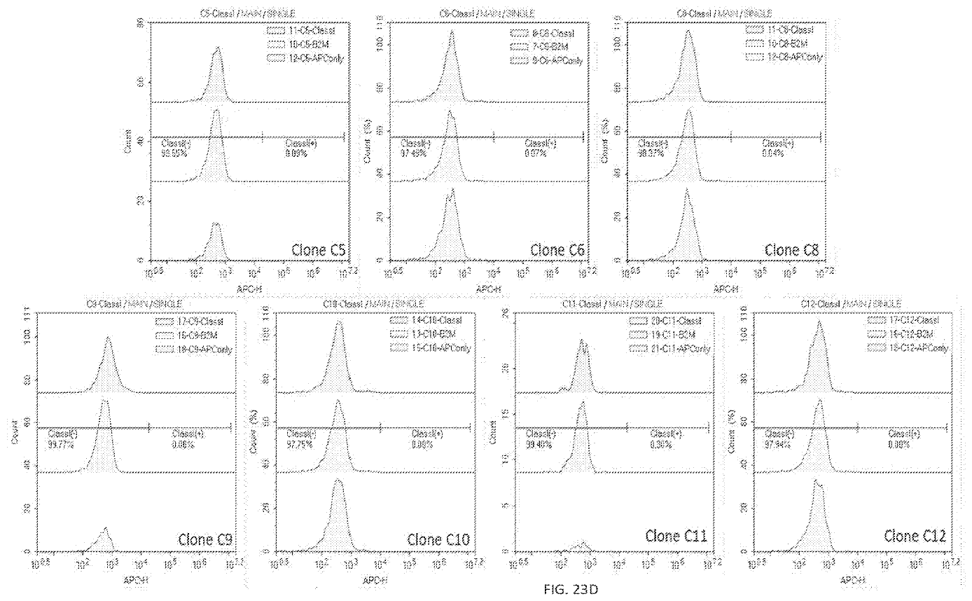

The steps of Porcine Donor B2M Humanization include: (1) A knockout of the porcine B2M Copy 1 and Copy 2 using a large fragment deletion at exon 2; (2) Insertion of B2M from a designated human donor was placed into the Rosa26 safe harbor site. Light grey is used to indicate a knockout or fragdel has occurred, dark grey is representative of a human donor knock-in of the gene. The pB2M KO Guide RNA Sequence and Cut Location is illustrated in FIG. 19. The specific guide RNAs are complexed together with the sp Cas9 to form a ribonucleoprotein (RNP). RNPs are then delivered to the cells via the optimized electroporation setting identified using a 200 point optimization. Copy 1 and Copy 2 of porcine B2M is knocked out using a large fragment deletion at exon 2. FIGS. 20A and 20B shows the map of Porcine B2M Copy 1 & 2, including exon 2, where a 9 base pair (bp) edit was inserted to knock out Porcine B2M Copy 1 and 1 base pair (bp) edit was inserted to knock out Porcine B2M Copy 2. Sanger Sequencing confirmed the 9 base pair (bp) and 1 base pair (bp) edits. FIGS. 21 and 22 show the results of the viability of the PAM clone cells and B2M expression in the clones, respectively. Clones A2 and A5 were selected as successful clones with least pB2M expression.

Viability of the PAM clone cells determined using trypan blue dye exclusion method using the Invitrogen Countess 3 instrument. Cells are incubated with trypan blue and placed on manufacturer's slides and inserted into the instrument, where dead cells take up the dye and are colored blue and viable cells are not blue. The instrument provides a readout of the percent viability. 85% of the clones (29/34) were higher than 88% viable prior to cryopreservation.

β2M protein expression in the genetically modified clones was assessed using quantitative sandwich enzyme immunoassay technique using a Pig Beta-2-Microglobulin ELISA kit. Methods for detection of pB2M in cell lysates of modified PAM clones and WT PAM by ELISA. Cells were cultured 80% confluency in 24-well plate. Cells were washed with 500 μL ice-cold 1×PBS two times. 200-300 μL ice-cold 1×PBS buffer was added into the wells. Adherent cells were scraped off the dish using a cold plastic cell scraper and transferred into a pre-cooled microfuge tube. After two freeze-thaw cycles at −80° C. were performed to break the cell membranes, the lysates were then centrifuged for 5 minutes at 5000×g at 4° C. Total protein concentration in each lysate was determined using the BioTek Take 3 Micro-Volume Plate and assayed immediately in ELISA experiment. Quantitative sandwich ELISA experiment is performed by following the manufacturer protocol. Briefly, antibody specific for pβ2M has been pre-coated onto a microplate. Standards and samples are pipetted into the wells and incubated 2 hours at 37° C. After removing any unbound substances, a biotin-conjugated antibody specific for β2M is added to the wells and incubated 2 hours at 37° C. After washing, avidin conjugated Horseradish Peroxidase (HRP) is added to the wells and incubated for an hour. Following a wash to remove any unbound avidin-enzyme reagent, a substrate solution is added to the wells. The color development is stopped, and the intensity of the color is measured at 450 nm and 570 nm.

Flow Histograms for cell surface B2M and SLA Class I molecules. Phenotyping analysis of porcine alveolar macrophages (PAM) from Wild Type (WT) and genome-edited cells suggests the complete SLA Beta-2-Microglobulin (B2M) knock-out clones were a success. Cells were activated for 48 hours with 100 ng/mL IFN-γ. The cells were stained for SLA Beta-2-Microglobulin. Methods for phenotype for expression of SLA-I and pB2M. Cells of each B2M modified clone were spun down and buffer/medium is removed from the wells. Master mix is prepared in flow buffer using 10 μg/mL monoclonal antibodies for SLA-class I and pβ2M per well. 100 μL staining buffer was transferred into the wells. Cells were mixed by gentle pipette up-down and Incubated 30 minutes at 4° C. Cells were spun down at ˜-300×g for 3 minutes and washed 2× using 200 μL flow buffer. Cells were stained with 10 μg/mL secondary antibody solution in flow buffer for 30 min at 4° C. Cells were washed two times using 200 μL flow buffer and resuspend in 200 μL 0.5% PFA containing MACS buffer. Data was acquired using Novacyte Flow cytometry

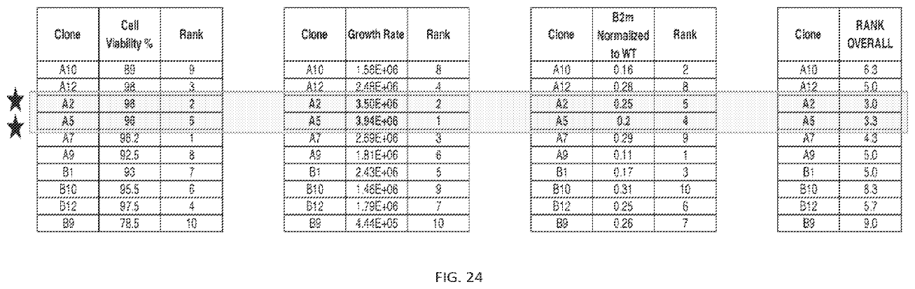

B₂M and SLA Class I expression levels detected using relative MFI values obtained from cell surface staining by flow cytometry. A total of 36 clones were thawed and cultured, 3 did not grow and were not phenotyped. Of those phenotyped, 25 clones were SLA Class I and B₂M negative out of a total of 33 clones. Cytosolic B₂M concentrations in cell lysates, identified by an ELISA assay, were between 110 ng/mL and 1112 ng/mL suggesting existence of translated protein. However, whether the expressed protein was intact or functional is unknown. Cells that met minimum criteria (MFI ≤1 and ELISA <0.5) were ranked from best (1) to worst (10) in the three categories as illustrated in FIG. 24. The two clone lines with the best average rank of these three parameters were chosen (highlighted). An ideal cell would have high viability and growth rate and low B₂M expression compared to the Wild Type (WT).

Porcine B2M Copy 1 & 2 Knock-out. Phenotyping analysis of porcine alveolar macrophages (PAM) from Wild Type (WT) and genome-edited cells suggests the complete SLA Beta-2-Microglobulin (B2M) knock-out clones were a success. Cells were activated for 48 hours with 100 ng/mL IFN-γ. The cells were stained for SLA Beta-2-Microglobulin. Report Number XLB407.

Porcine B₂M Copy 1 & 2 Fragment Deletions. Porcine B₂M. Simulating Agarose Gel Electrophoresis in SnapGene comparing Sanger sequencing from the WT PAM cell to the porcine b2m fragdel edits on PAM cell A2. Within the perspective gel model, lane 1 is porcine B2m sequence, lane 2 is A2 copy 1 exons only, lane 3 is A2 copy 2 exons only.

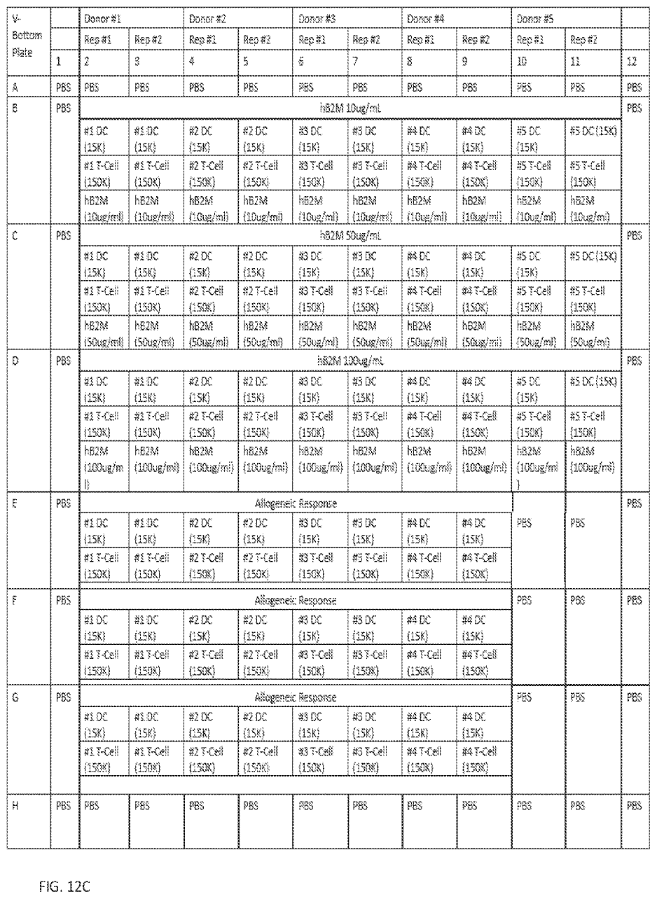

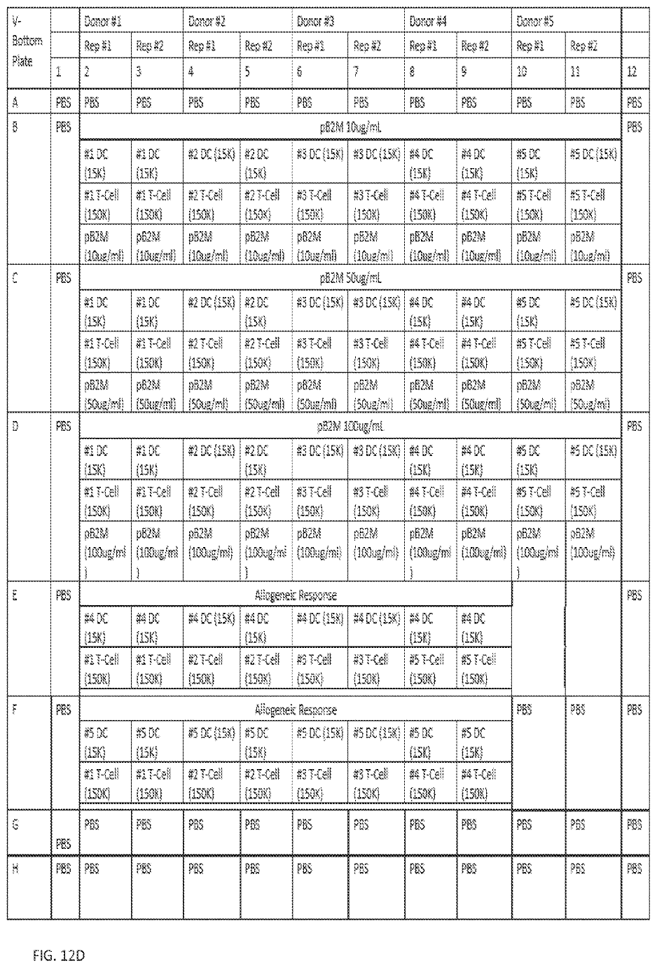

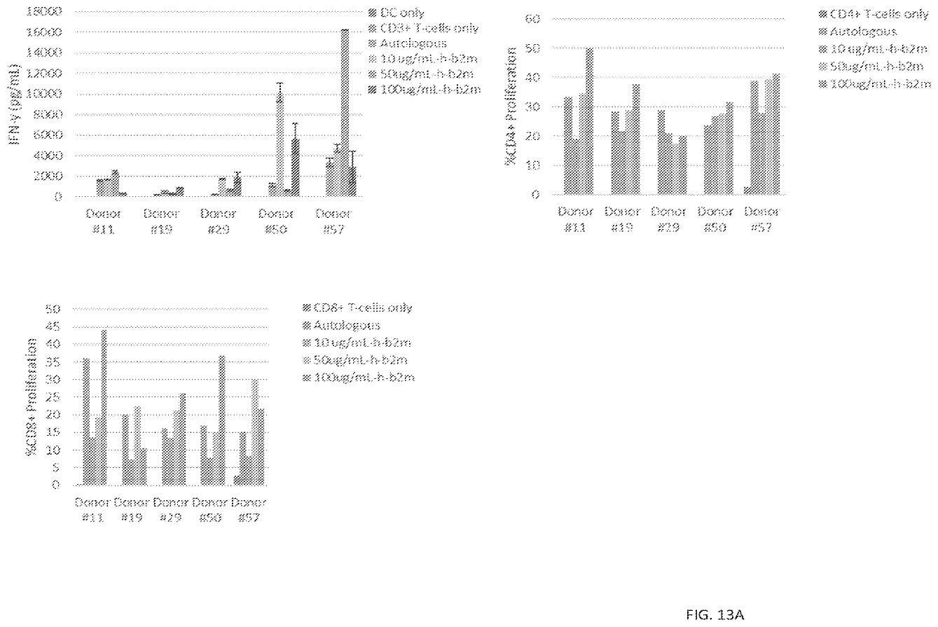

A non-GLP study is conducted to measure CD3(+) T-cells response by means of IFN-γ production and T-cell proliferation to porcine whole protein Beta-2 microglobulin processed and presented by professional antigen presenting dendritic cells. Keyhole Limpet Hemocyanin (KLH) and human Beta-2 microglobulin recombinant protein with 6×His-tag at C-terminal are used as controls. The plate design for MLR Assay is demonstrated in FIGS. 12A-12D. The objective of this non-GLP study is to measure CD3(+) T-cells response by means of IFN-γ production and T-cell proliferation to porcine whole protein Beta-2 microglobulin processed and presented by antigen presenting dendritic cells. Keyhole Limpet Hemocyanin (KLH) and human Beta-2 microglobulin recombinant protein with 6×His-tag at C-terminal used as controls.

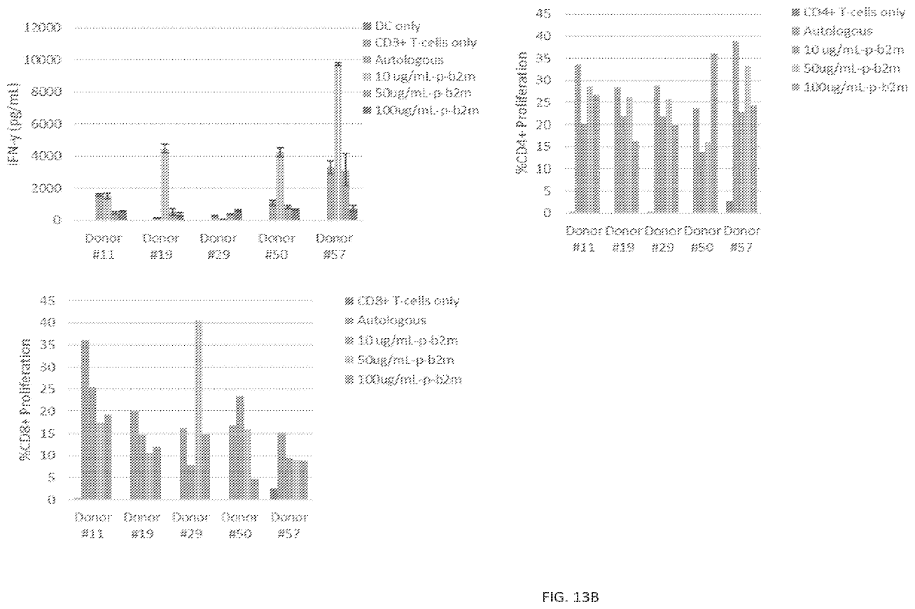

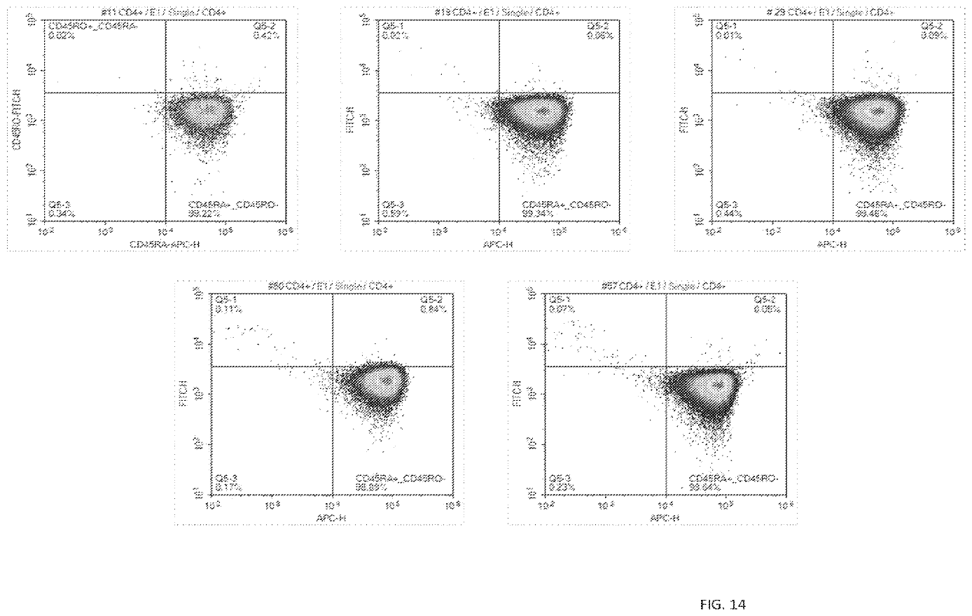

The immunogenicity responses of human CD3(+) T cells (Donor #11, #19, #29, #50 and #57) co-cultured with autologous human B2M loaded/unloaded dendritic cells are presented as CD4(+) T-cell (A), CD8(+) T-cell (B) proliferations and IFN-γ production(C). Human PBMCs from five different IRB donors (Donor #11, #19, #29, #50, and #57) sourced by Xeno Diagnostics, LLC through its Institutional Review Board (IRB) program were used in this study as shown in FIG. 13A and also for porcine dendritic cells as shown in FIG. 13B. Blood samples were collected, PBMCs were isolated by Ficoll density gradient centrifugation and cryopreserved. Prior to use, the cryopreserved PBMCs were thawed and donor monocytes were isolated using a Monocyte Isolation Kit (Stem Cells Technologies). Dendritic cells were generated using an animal component-free (ACF), serum-free medium, ImmunoCult™-ACF DC (Stem Cell Technologies). Dendritic cells were loaded with protein antigens (1) Keyhole Limpet Hemocyanin (KLH) (2) Recombinant Human Beta-2 Microglobulin (B2M) with 6×His-tag and (3) Recombinant Porcine Beta-2 Microglobulin with 6×His-tag in the presence of maturation supplement. Donor #11, #19, #29, #50 and #57 PBMCs were thawed and CD3(+) T-cells (untouched/negatively selected) were isolated using a CD3(+) T-cell isolation kit (StemCell Technology). CD3(+) T-cells were labeled using CellTrace™ Violet (CTV) Cell Proliferation Kit (Invitrogen) and were co-cultured with autologous activated antigen loaded DCs or allogeneic antigen unloaded DCs in serum-free Optimizer medium (Gibco) supplemented with Optimizer T-cell expansion supplement (Gibco), and 2 mM GlutaMAX (Gibco) for seven days to measure cumulative CD3+ T-cell response. CTV labeled CD3+ T-cell proliferation was analyzed by flow cytometry on Day 7 using 7AAD, CD3-APC (Clone UCHT1, Biolegend), CD4-PE/Cy7 (Clone RPA-T4, Biolegend) and CD8-PE (Clone SKI, Biolegend) antibodies. Cytokine production was measured on Day 7 using MagPix™ Milliplex (Luminex™ technology).

Porcine B2M Copy 1 & 2 Knock-out Naive T-cells Enrichment. PBMC from IRB donors were thawed and prepared in PBS, 2% FBS and 1 mM EDTA according to manufacturer's instructions at a concentration of 5×10⁷ cells/ml. EasySep™ Human Naive CD4+ T Cell Isolation Cocktail 11 (StemCell Technologies), 50 μL is added and mixed with the cells, incubated for 5 minutes. RapidSpheres 50 μL are added, add 2.5 mL of PBS media, gently mix, place into the magnet holder for 3 minutes. Decant isolated cells. It appears that we are at the limits of detection of the assay and very few T cells are responding to either human or porcine B2M. Off the shelf antibodies today cannot determine the difference between hβ2M and pβ2M; therefore custom, species-specific antibodies are required.

### Human-β2M Knockin at Porcine Rosa26 Safe Harbor Site

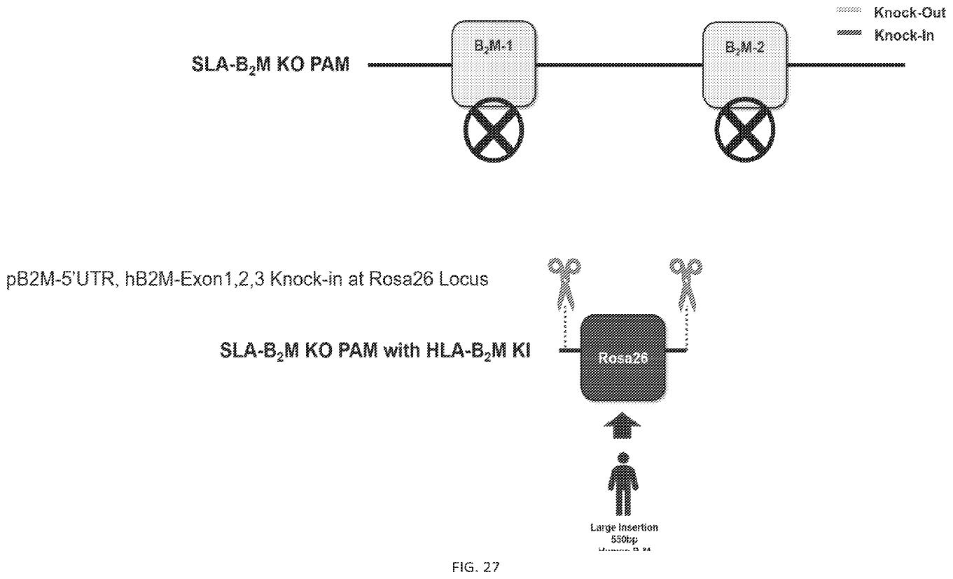

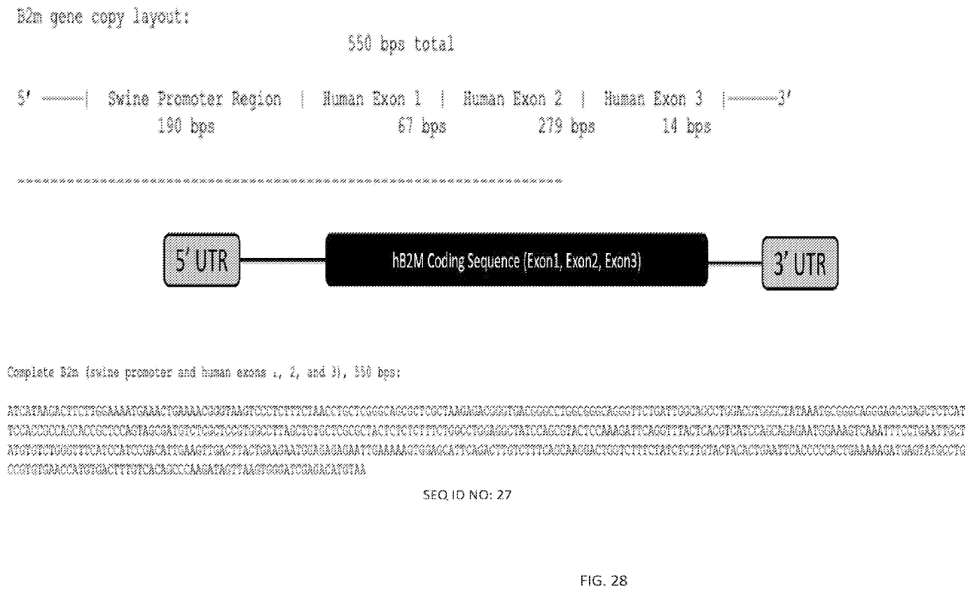

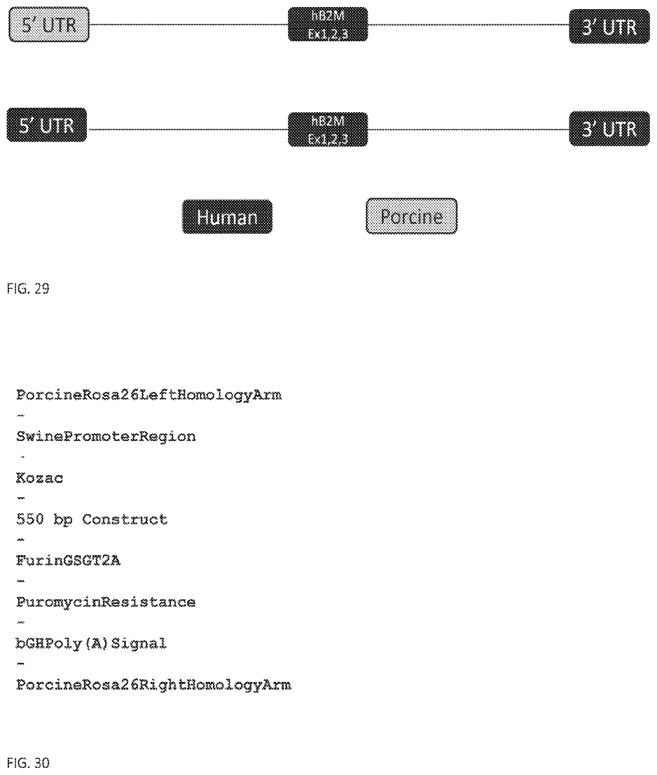

As demonstrated in FIG. 27, insertion of B2M from a designated human donor was placed into the Rosa26 safe harbor site. Light grey is used to indicate a knockout or fragdel has occurred, dark grey is representative of a human donor knock-in of the gene. hB2M KI Construct for Insertion into ROSA26 Site is illustrated in FIG. 30. Of the construct for insertion, FIG. 16 illustrates the schematic of the porcine promoter region. The sequences for the promoter, Exon 1 and Exon 2 of Rosa26 region from the PAM cell line is further illustrated in FIG. 28. As illustrated in FIG. 29, different permutations of hB2M KI Construct for Insertion into ROSA26 site are possible. A complete B2m sequences comprising porcine promoter and human exons 1,2,3 upon Human B2M KI at Rosa26 safe harbor site is illustrated in FIG. 28. As clearly demonstrated in FIG. 27, a large insertion (550 bp) of Human B2M sequences were inserted to Rosa 26 locus of porcine B2M gene.

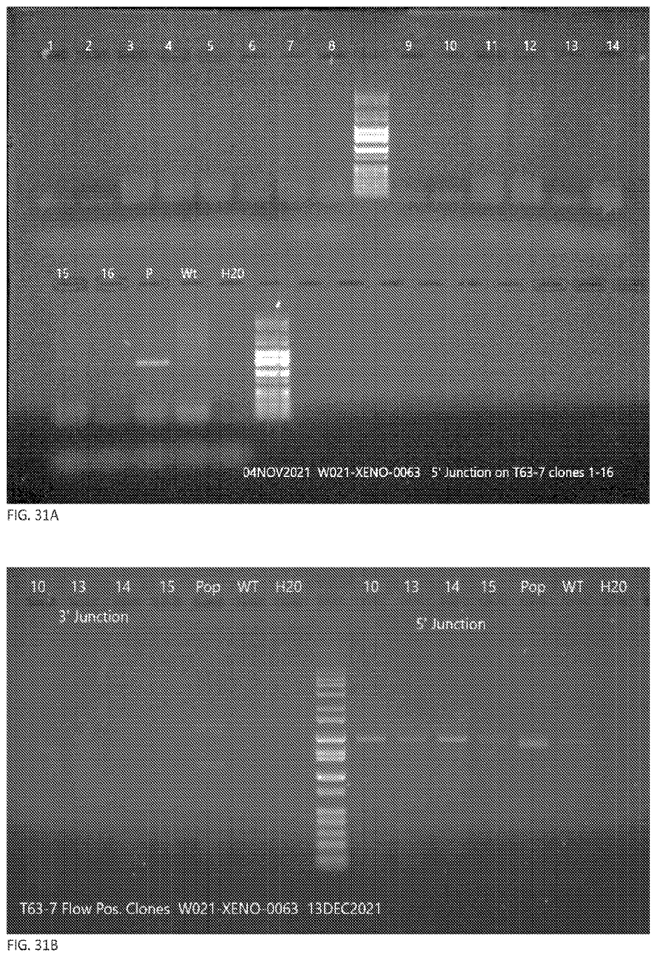

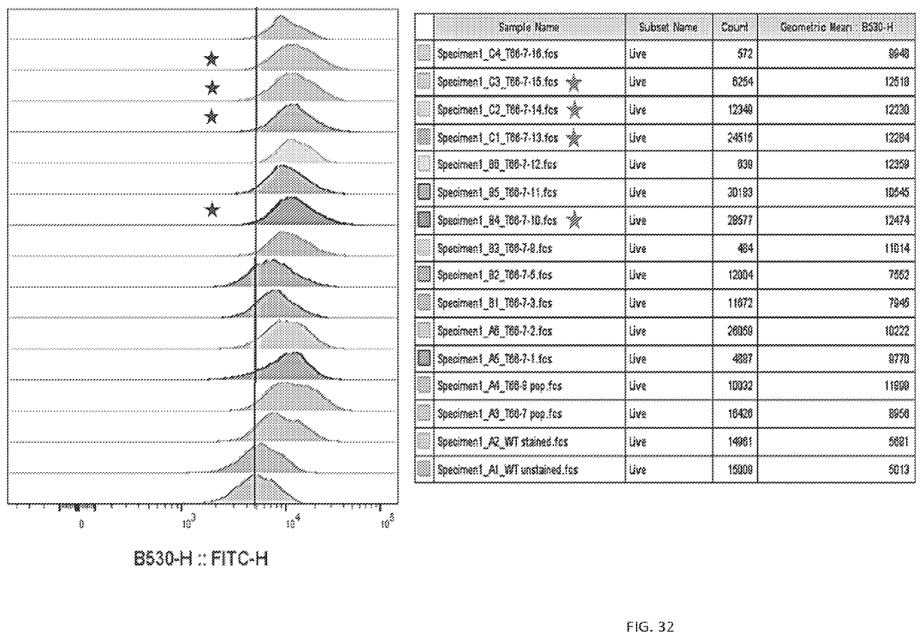

Human B2M KI at Porcine Rosa26 safe harbor site. FIGS. 31A and 31B shows junction PCR attempt on clones that are puro resistant. Clones show modest B2M expression, with Clones 10, 13, 14, and 15 showing best expression. As shown in FIG. 32, screening for cell surface expression of hB2M on cell clones was detected using a specific anti-human B2M monoclonal antibody, B530-H using flow cytometry. Four clones identified with stars show the strongest hB2M on the cell surface supporting the presence of a functional hB2M gene.

### Human and Porcine B2M Cross-Reactive Epitope Groups (CREGs) Predictive Analysis

To assess whether a B2M mismatch within a CREG group may result in a better outcome than a mismatch outside CREG groups, we analyzed validated outcomes data against known amino acid characteristics. Cross-reactions are due to specific amino acid (aa) linear or conformational combinations designated as triplets and eplets shared by different proteins. Based on eplet, triplet or simple electrochemical distances of molecules between different species (or recipient and donor), the humoral response to different antigenic aa sequence of a protein can be predicted.

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations are not intended to convey that the claimed subject matter requires features or combinations of features other than those expressly recited in the claims. Accordingly, the scope of this disclosure is intended to include all modifications and variations encompassed within the spirit and scope of the following appended claims.

1. A method of generating a genetically reprogrammed non-human donor animal comprising:
a) obtaining a multipotent or pluripotent cell from a wild-type non-human donor animal, wherein said multipotent or pluripotent cell comprise a porcine genome comprising copy 1 and copy 2 of a porcine β2-microglobulin (pβ2M) gene;
b) inactivating copy 1 and copy 2 of the pβ2M gene in the porcine genome;
c) forming a genomic sequence construct that comprises a) a nucleic acid sequence encoding a 5′ UTR upstream region of a hβ2M gene or a pβ2M gene including its promoter sequence and b) a nucleic acid sequence encoding a hβ2M protein;
d) inserting the genomic sequence construct of step c) into a safe harbor locus of the porcine genome to form a genetically reprogrammed cell;
e) generating an embryo from the genetically reprogrammed cell;
f) transferring the embryo into a surrogate animal; and
g) growing the transferred embryo to produce the genetically reprogrammed non-human donor animal as offspring of said surrogate animal.

2. A method of generating a genetically reprogrammed non-human donor animal comprising:
a) obtaining a multipotent or pluripotent cell from a wild-type non-human donor animal, wherein said multipotent or pluripotent cell comprise a porcine genome comprising copy 1 and copy 2 of a porcine β2-microglobulin (pβ2M) gene;
b) inactivating copy 1 and/or copy 2 of the pβ2M gene in the porcine genome;
c) forming a genomic sequence construct that comprises a) a nucleic acid sequence encoding a 5′ UTR upstream region of a hβ2M gene or a pβ2M gene including its promoter sequence and b) a nucleic acid sequence encoding a hβ2M protein;
d) performing scarless exchange of a nucleotide acid sequence encoding copy 1 and/or copy 2 of the pβ2M gene in the porcine genome with the genomic sequence construct of step c) to form a genetically reprogrammed cell;
e) generating an embryo from the genetically reprogrammed cell;
f) transferring the embryo into a surrogate animal; and
g) growing the transferred embryo to produce the genetically reprogrammed non-human donor animal as offspring of said surrogate animal.

3. The method of claim 1, wherein:
i) a) is a (5′ UTR) upstream region of a hβ2M gene; or
ii) a) is a (5′ UTR) upstream region of a pβ2M gene.

4. The method of claim 1, wherein the multipotent or pluripotent cell is a fibroblast, mesenchymal stem cell, bone marrow cell, zygote, induced pluripotent stem cell (IPSC), or germ-line cell.

5. The method of claim 1, further comprising making genetic alterations to the porcine genome that reduce natural immunologic response in a recipient after xenotransplantation.

6. The method of claim 5, wherein the genetic alterations comprise at least one of:
a) genes encoding alpha-1,3 galactosyltransferase (GalT), cytidine monophosphate-N-acetylneuraminic acid hydroxylase (CMAH), and beta-1,4-N-acetylgalactosaminyltransferase (B4GALNT2) are disrupted such that the genetically reprogrammed porcine donor lacks functional expression of surface glycan epitopes encoded by said genes;
b) reprogramming the porcine genome such that endogenous exon and/or intron regions of the wild-type porcine donors' Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DQB and any combination thereof, that are disrupted, silenced or otherwise not functionally expressed on (95%) of extracellular surfaces achieved through specific combinations of precise, site-directed mutagenic substitutions or modifications;
c) reprogramming the porcine genome such that endogenous exon and/or intron regions of the wild-type porcine donor's PD-L1, CTLA-4, EPCR, TBM, TFPI, and/or MIC-2, and any combination thereof, that are humanized via reprogramming through specific combinations of precise, site-directed mutagenic substitutions or modifications with synthetic nucleotides from orthologous exons of a known human PD-L1, CTLA-4, EPCR, TBM, TFPI, and MIC-2 from the human captured reference sequence, designed from the human captured reference sequence and which minimizes collateral genomic disruptions and ideally results in no net gain or loss of total numbers of nucleotides and avoids genomic organizational disruption and that render the donor animal's cells, tissues, and organs tolerogenic when transplanted into a human without sacrificing the natural immune function of the PD-L1, CTLA-4, EPCR, TBM, TFPI, and MIC-2 proteins; or
d) reprogramming the porcine genome such that endogenous exon and/or intron regions of the wild-type porcine donor's Major Histocompatibility Complex corresponding to exon regions of SLA-1, SLA-2, SLA-3, SLA-6, SLA-7, SLA-8, SLA-DRA, SLA-DRB, SLA-DQA, and/or SLA-DOB, and any combination thereof, that are reprogrammed through specific combinations of precise, site-directed mutagenic substitutions or modifications with synthetic nucleotides from orthologous exons of a known human HLA-A, HLA-B, HLA-C, HLA-E, HLA-F, HLA-G, HLA-DRA, HLA-DRB, HLA-DQA, and/or HLA-DQB from the human captured reference sequence, designed from the human captured reference sequence and which minimizes collateral genomic disruptions and ideally results in no net gain or loss of total numbers of nucleotides and avoids genomic organizational disruption and that render the donor animal's cells, tissues, and organs tolerogenic when transplanted into a human without sacrificing the natural immune function of the SLA-1, SLA-2, SLA-3, SLA-6, SLA-7, SLA-8, SLA-DRA, SLA-DRB, SLA-DQA, and/or SLA-DQB proteins.

7. The method of claim 1, wherein the porcine genome is reprogrammed to comprise at least one stop codon selected from TA, TAG, and TGA, or a sequential combination of two or three of said stop codons.

8. The method of claim 7, wherein the reprogrammed genome comprises said at least one stop codon or said combination of two or three of said stop codons more than 70 base pairs downstream from the promoter of a gene or genes to be silenced such that the wild-type porcine donor gene lacks functional expression of said gene or genes.

9. The method of claim 1, wherein the wild-type non-human donor animal was reared in a Designated Pathogen Free (DPF) environment and is free of at least the following zoonotic pathogens:
(i) Ascaris species, cryptosporidium species, Echinococcus, Strongyloids sterocolis, and Toxoplasma gondii in fecal matter;
(ii) Leptospira species, Mycoplasma hyopneumoniae, porcine reproductive and respiratory syndrome virus (PRRSV), pseudorabies, transmissible gastroenteritis virus (TGE)/Porcine Respiratory Coronavirus, and Toxoplasma gondii by determining antibody titers;
(iii) Porcine Influenza;
(iv) the following bacterial pathogens as determined by bacterial culture: Bordetella bronchiseptica, Coagulase-positive staphylococci, Coagulase-negative staphylococci, Livestock-associated methicillin resistant Staphylococcus aureus (LA MRSA), Microphyton and Trichophyton spp.;
(v) Porcine cytomegalovirus; and
(vi) Brucella suis.

10. A genetically modified porcine donor animal wherein copy 1 and copy 2 of the β2M gene of the porcine donor animal genome are modified and/or inactivated by the method of claim 1.

11. A donor porcine donor animal cell, tissue or organ for xenotransplantation obtained from a genetically modified porcine donor animal of claim 10.

12. The donor porcine donor animal cell, protein, tissues, or organ for xenotransplantation of claim 11, wherein the cells from the genetically modified porcine donor animal when co-cultured with human peripheral blood mononuclear cells (PBMCs) induce a lower CD8+ T cell immune response as compared to cells from said non-genetically modified counterpart pig, as measured by an in vitro mixed lymphocyte reaction assay.

13. A method of producing a donor porcine donor animal cells, tissues or organs for xenotransplantation, comprising producing the genetically modified porcine donor animal of claim 10, and harvesting a cell, tissue, or organ from the genetically modified porcine donor animal.

14. A donor porcine donor animal cell, protein, tissue or organ for xenotransplantation produced from the method of claim 13, wherein the cells from the genetically modified porcine donor animal when co-cultured with human peripheral blood mononuclear cells (PBMCs) induce a lower CD8+ T cell immune response as compared to cells from said non-genetically modified counterpart pig, as measured by an in vitro mixed lymphocyte reaction assay.
